SEQUENCE LISTING (9 sequences)

SEQ ID NO: 1 - RNA, 40 nt, synthetic construct
  source 1..40: mol_type = other RNA
aaaaaaaaaaaaaaaaaaaaaaaaaaaaaaaaaaaaaaaa

SEQ ID NO: 2 - RNA, 40 nt, synthetic construct
  source 1..40: mol_type = other RNA
aaaaaaaaaaaaaaaaaaaaaaaaaaaaaaaaaaaaaaac

SEQ ID NO: 3 - RNA, 40 nt, synthetic construct
  source 1..40: mol_type = other RNA
aaaaaaaaaaaaaaaaaaaaaaaaaaaaaaaaaaaaaaag

SEQ ID NO: 4 - RNA, 40 nt, synthetic construct
  source 1..40: mol_type = other RNA
aaaaaaaaaaaaaaaaaaaaaaaaaaaaaaaaaaaaaaat

SEQ ID NO: 5 - RNA, 60 nt, synthetic construct
  source 1..60: mol_type = other RNA
aaaaaaaaaaaaaaaaaaaaaaaaaaaaaaaaaaaaaaaaaaaaaaaaaaaaaggcccct

SEQ ID NO: 6 - RNA, 40 nt, synthetic construct; identical to SEQ ID NO: 1
  source 1..40: mol_type = other RNA

SEQ ID NO: 7 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 33 bp, synthetic construct
  source 1..33: mol_type = other DNA
  5'UTR 1: standard_name = P1, monophosphate
  misc_binding 25: bound_moiety = (bio)2, biotin
ctgtctcttatacacatctgacgctgccgacga

SEQ ID NO: 9 - DNA, 43 bp, synthetic construct
  source 1..43: mol_type = other DNA
tcgtcggcagcgtcagatgtgtataagagacagtttttttttt

(19) United States
(12) Patent Application Publication
(10) Pub. No.: US 2024/0309447 A1
(43) Pub. Date: Sep. 19, 2024

(54) METHODS AND KITS FOR PREPARING A SAMPLE COMPRISING ONE OR MORE RNA MOLECULES FOR SEQUENCING

(71) Applicant: The Hong Kong University of Science and Technology, Hong Kong (CN)

(72) Inventors: Yi KUANG, Hong Kong (CN); Zhenghua LIANG, Hong Kong (CN); Angela Ruohao WU, Hong Kong (CN)

(21) Appl. No.: 18/601,981

(22) Filed: Mar. 11, 2024

Related U.S. Application Data
(60) Provisional application No. 63/490,526, filed on Mar. 16, 2023.

(51) Int. Cl. C12Q 1/6874; C12Q 1/6806

(52) U.S. Cl. CPC C12Q 1/6874; C12Q 1/6806; C12Q 2600/158

(57) ABSTRACT

One example embodiment is a method for preparing a sample including one or more RNA molecules, including the steps of: (i) reacting the sample with a reaction mixture including a poly(A) polymerase and one or more predefined nucleotide analogues to add a first sequence including one or more predefined nucleotide analogues to individual 3′-end of the one or more RNA molecules, such that one or more first tailed RNA is formed; and (ii) adding a plurality of adenosine triphosphates (ATP) to the reaction mixture, such that the first tailed RNA is reacted with a plurality of adenosine triphosphates (ATP) to add a second sequence including a plurality of adenine nucleotides to individual 3′-end of the first tailed RNA, such that one or more second tailed RNA is formed. Other example embodiments are described herein. In certain embodiments, the disclosed methods and kits are quick, simple and easy to operate.

### CROSS-REFERENCE TO RELATED APPLICATION

This application claims benefit under 35 U.S.C. § 119(e) of U.S. Provisional application having Ser. No. 63/490,526 filed Mar. 16, 2023, the entire contents of which is hereby incorporated by reference herein.

### REFERENCE TO SEQUENCE LISTING

This application contains a sequence listing which has been submitted electronically in ST.26 (xml) format and is hereby incorporated by reference in its entirety. Said ST.26 copy, created on 22 Dec. 2023, is named “H001.0000.003.NPRUS seq list.xml” and is 10 kilobytes in size.

### FIELD OF INVENTION

This application relates to methods for preparing a sample comprising one or more RNA molecules. In particular, this application relates to methods and kits for preparing a sample comprising one or more RNA molecules for sequencing such as direct RNA sequencing.

### BACKGROUND OF INVENTION

Direct RNA sequencing reads native RNA strands by identifying the characteristic current disruption caused by different nucleotides as the RNA molecule passes through the nanopore. Direct RNA sequencing is important due to its ability to analyze the RNA 3′ tails and RNA modifications, which is essential to mRNA biology studies into disease development, cell development, and others.

The conventional sample preparation methods for direct RNA sequencing of mRNA use dT adapter to ligate mRNAs for sequencing, which often lead to difficulties and low efficiencies in sequencing the mRNAs with 3′ termini containing non-A bases. There is a need for improved methods and kits for preparing a sample comprising one or more RNA molecules, especially for direct RNA sequencing.

### SUMMARY OF INVENTION

In the light of the foregoing background, in certain embodiments, it is an object to provide improved methods and kits for preparing a sample comprising one or more RNA molecules for RNA sequencing.

Accordingly, in one aspect, provided herein is a method for preparing a sample including one or more RNA molecules, including the steps of: (i) reacting the sample with a reaction mixture including a poly(A) polymerase and one or more predefined nucleotide analogues to add a first sequence including one or more predefined nucleotide analogues to individual 3′-end of the one or more RNA molecules, such that one or more first tailed RNA is formed; and (ii) adding a plurality of adenosine triphosphates (ATP) to the reaction mixture, such that the one or more first tailed RNA is reacted with the plurality of adenosine triphosphates (ATP) to add a second sequence including a plurality of adenine nucleotides to individual 3′-end of the one or more first tailed RNA, such that one or more second tailed RNA is formed.

In some embodiments, provided is a method for sample preparation for direct RNA sequencing, including the steps of: (i) reacting the sample including one or more RNA molecules with a reaction mixture including a poly(A) polymerase and one or more predefined nucleotide analogues to add a first sequence including one or more predefined nucleotide analogues to individual 3′-end of the one or more RNA molecules, such that one or more first tailed RNA is formed; and (ii) adding a plurality of adenosine triphosphates (ATP) to the reaction mixture, such that the one or more first tailed RNA is reacted with the plurality of adenosine triphosphates (ATP) to add a second sequence including a plurality of adenine nucleotides to individual 3′-end of the one or more first tailed RNA, such that one or more second tailed RNA is formed.

In another aspect, provided is a kit for preparing a sample including one or more RNA molecules for sequencing, including: (a) a poly(A) polymerase; (b) one or more predefined nucleotide analogues; and (c) a plurality of adenosine triphosphates (ATP), (d) and optionally, d) a ligase; e) a sequencing adapter; and/or f) one or more buffer solutions.

Other example embodiments will be described below.

### Advantages

There are many advantages of the present disclosure. In some embodiments, the disclosed sample preparation methods (also referred to as “one-pot 3′ extension” in some embodiments) use poly(A) polymerase to first add a first sequence (also referred to as “oligo nucleotide analogue sequence” in some embodiments) including one or more predefined nucleotide analogues that are chemically distinct from native nucleotides to one or more RNA molecules in the sample, and then add a second sequence (also referred to as “oligo(A) sequence” in some embodiments) including one or more adenine (A) nucleotides. As such, in some embodiments, the first sequence can serve as a marker to locate the last base of the 3′ tail of individual RNA molecule in sequencing, so the intact 3′ tail sequence of the individual RNA molecule can be correctly mapped.

In some embodiments, the disclosed methods enable all RNA molecules (including RNA molecules with 3′ termini containing A or non-A bases) in the sample to unbiasedly anneal to the sequencing adapter (also referred to as “dT adapter” in some embodiments), increasing mRNA enrichment yield for sequencing. The ability of the disclosed methods to capture RNA molecules with 3′ termini containing non-A bases is a significant improvement over the conventional methods, as NGS sequencing data have revealed that not all of the poly(A) tails of mRNAs contain only As, and especially there are a lot of non-A nucleotides in the 3′ terminus. Non-A nucleotide in the tail is also dynamic and found in oocyte maturation, virus infection, and other biological processes. The non-A-containing tails play important roles in mRNA regulation.

In some embodiments, the disclosed methods and kits are compatible with standard sample preparation protocols for RNA sequencing, without the need of switching the existing equipment or reagent kits. In some embodiments, the disclosed methods are quick, simple and easy to operate.

### BRIEF DESCRIPTION OF FIGURES

FIG. 1 shows a workflow of an example method for preparing a sample comprising one or more RNA molecules for sequencing according to an example embodiment.

FIG. 2 shows the Urea-PAGE result of reacted RNA products after adding different nucleotide analogues to the 3′ termini of the model RNA by poly(A) polymerase-based extension according to example embodiments.

FIG. 3 shows the Urea-PAGE result of reacted RNA products with different incubation time for adding 2-Amino-6-Cl-purine-rTP/ATP to the 3′ termini of RNA by poly(A) polymerase-based extension according to example embodiments.

FIGS. 4A, 4B and 4C show the Urea-PAGE results of reacted RNA products after adding different nucleotide analogues to the 3′ termini of RNAs with C terminal base (FIG. 4A), G terminal base (FIG. 4B) and U terminal base (FIG. 4C) respectively by poly(A) polymerase-based extension according to example embodiments.

FIG. 5 is a graph produced by Fragment Analyzer showing reacted RNA products after adding 2-Amino-6-Cl-purine-rTP to the 3′ termini of model mRNAs by poly(A) polymerase according to example embodiments.

FIG. 6 shows the Urea-PAGE result of reacted RNA products with different incubation time for adding adenine nucleotides (oligo A) to the 3′ termini of first tailed RNA carrying oligo 2-Amino-6-Cl-purine-rTP sequence according to example embodiments.

FIG. 7 shows the Urea-PAGE result of mRNA samples treated with dT adapter ligation with or without prior one-pot 3′ extension reaction according to example embodiments.

FIG. 8 shows the raw current signal of Nanopore direct RNA sequencing of RNA samples after one-pot 3′ extension according to an example embodiment.

FIGS. 9A and 9B are graphs produced by Fragment Analyzer showing reacted RNA products with different incubation time and different nucleotide analogue concentrations for adding nucleotide analogues to the 3′ termini of UnaG-120A mRNA according to example embodiments.

FIG. 9C is a graph produced by Fragment Analyzer showing reacted RNA products using different nucleotide analogue concentrations for adding nucleotide analogues to the 3′ termini of EGFP-40A and Cluc-53ACCGGGGU mRNA according to example embodiments.

FIG. 10 is a graph produced by Fragment Analyzer showing reacted RNA products after adding 2-Amino-ATP, 2-Amino-6-Cl-purine-rTP, or a combination of 2-Amino-ATP and 2-Amino-6-Cl-purine-rTP to the 3′ termini of model RNA by poly(A) polymerase according to example embodiments.

### DETAILED DESCRIPTION

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For example, alternate embodiments of “a composition comprising A, B, and C” would be “a composition consisting of A, B, and C” and “a composition consisting essentially of A, B, and C.” Even if the latter two embodiments are not explicitly written out, this disclosure/application includes those embodiments. Furthermore, it shall be understood that the scopes of the three embodiments listed above are different.

For the sake of clarity, “comprising”, including, and “containing”, and any related forms are open-ended terms which allows for additional elements or features beyond the named essential elements, whereas “consisting of” is a closed end term that is limited to the elements recited in the claim and excludes any element, step, or ingredient not specified in the claim.

As used herein, the singular forms “a”, “an” and “the” are intended to include the plural forms as well, unless the context clearly indicates otherwise. Where a range is referred in the specification, the range is understood to include each discrete point within the range. For example, 1-7 means 1, 2, 3, 4, 5, 6, and 7.

As used herein, the term “about” is understood as within a range of normal tolerance in the art and not more than ±10% of a stated value. By way of example only, about 50 means from 45 to 55 including all values in between. As used herein, the phrase “about” a specific value also includes the specific value, for example, about 50 includes 50.

As used herein, the term “variant sequence” refers to a nucleic acid or polypeptide sequence that displays certain degree of identity to a reference or wild-type nucleic acid or polypeptide sequence, for example, at least 80%, at least 85%, at least 90%, at least 95%, at least 96%, at least 97%, at least 98% or at least 99%. For example, a variant sequence has one or more additions, deletions, insertions, and/or substitutions or other modifications when compared to a reference or wild-type sequence. Variant sequence also includes sequence of a functional homologue.

As used herein and in the claims, the term “polymerase” generally refers to an enzyme that is capable of adding one or more nucleotides or nucleotide analogues to the 3′ termini of an oligonucleotide, e.g. a RNA oligonucleotide or a RNA molecule. In some embodiments, a polymerase is an RNA polymerase. In some embodiments, a polymerase is a poly(A) polymerase capable of adding one or more nucleotides comprising an adenine (A) base or nucleotide analogues to the 3′ termini of an oligonucleotide, e.g. RNA oligonucleotide. In some embodiments, a poly(A) polymerase is capable of adding one or more nucleotide analogues to the 3′ termini of an oligonucleotide, e.g. RNA oligonucleotide, while retaining a high selectivity to nucleotides comprising an adenine (A) base. Examples of poly(A) polymerase include but are not limited to bacterial poly(A) polymerase (such as E. coli poly(A) polymerase), yeast poly(A) polymerase, and mammalian poly(A) polymerase (such as calf thymus poly(A) polymerase). As used herein and in the claims, the term “oligonucleotide” or “oligo” refers to a polymer of nucleotides or nucleotide analogues. An oligonucleotide may be naturally occurring or synthetic. In some embodiments, an oligonucleotide is an RNA oligonucleotide. In some embodiments, an oligonucleotide is a DNA oligonucleotide.

As used herein and in the claims, the term “nucleotide analogue” refers to a variant of naturally-occurring nucleotides, such as nucleotides comprising modifications in the sugar and/or base moieties.

As used herein and in the claims, the term “tail”, “tail sequence” and their derivatives refer to a nucleotide sequence that is added to the 3′ termini of a substrate RNA molecule, and the process of adding at least one nucleotide to a substrate RNA molecule is referred to herein as “tailing”.

As used herein and in the claims, the term “sequencing” refers to a technology that includes obtaining sequence information from one or more nucleic acid molecules by determining the identity and/or order of at least some nucleotides within each nucleic acid molecule. In some embodiments, the sequencing is RNA sequencing that sequences one or more RNA molecules. In some further embodiments, the sequencing is direct RNA sequencing that sequences one or more mRNA molecules.

As used herein, “direct RNA sequencing” refers to a technology that sequences one or more RNA molecules directly without going through amplification or reverse transcription. In some embodiments, direct RNA sequencing is performed using nanopore arrays (known as “Nanopore direct RNA sequencing”).

As used herein and in the claims, the term “RNA molecule” refers to a molecule that contains ribonucleotides. Examples of RNA molecules are messenger RNA, transfer RNA (tRNA), ribosomal RNA (rRNA), short interfering RNA (siRNA), short-hairpin RNA (shRNA), micro-RNA (miRNA), an isolated or synthetic RNA oligonucleotides.

As used herein, the terms “ligating”, “ligation” and their derivatives refer generally to the process for covalently linking two or more molecules together, for example covalently linking two or more nucleic acid molecules to each other.

As used herein, “ligase” and its derivatives, refers generally to any agent such as an enzyme capable of catalyzing the ligation of two substrate molecules.

As used herein and in the claims, the term “sequencing adapter” refers to refers to one or more oligonucleotides that can be ligated to a target nucleic acid molecule or fragments thereof for sequencing. In some embodiments, a sequencing adapter includes two oligonucleotides that at least have a complementary portion, forming an adapter that is double stranded at the complementary portion. In some embodiments, the two oligonucleotides of the sequencing adapter further include at least one mismatched portion. In some embodiments, the mismatched portion has at least one overhang.

As used herein, the term “enrich” means increasing the proportion of molecule target of interest among all molecules from a sample.

As used herein, the term “homolog” refers to a polypeptide that exhibits certain sequence identity with a reference or wild-type sequence and possess certain aspect of the reference or wild-type polypeptide's functionality.

In some embodiments, provided is a method for preparing a sample including one or more RNA molecules, including the steps of: (i) reacting the sample with a reaction mixture including a poly(A) polymerase and one or more predefined nucleotide analogues to add a first sequence including one or more predefined nucleotide analogues to individual 3′-end of the one or more RNA molecules, such that one or more first tailed RNA is formed; and (ii) adding a plurality of adenosine triphosphates (ATP) to the reaction mixture, such that the one or more first tailed RNA is reacted with a plurality of adenosine triphosphates (ATP) to add a second sequence including a plurality of adenine nucleotides to individual 3′-end of the one or more first tailed RNA, such that one or more second tailed RNA is formed.

In some implementations, step (i) and step (ii) above are performed sequentially in the same reaction mixture with the poly(A) polymerase. In step (ii), the plurality of adenosine triphosphates (ATP) is added directly in the reaction mixture which includes one or more first tailed RNA of step (i), without the need to purify the reaction mixture prior to step (ii). In some implementations, the resulting mixture is incubated at the same reaction condition as step (i). This makes the preparation steps easy to operate, while reducing the risk of RNA degradation by minimizing the processing steps. In some embodiments, the method does not comprise the step of purifying the one or more first tailed RNA prior to step (ii).

In some embodiments, the one or more predefined nucleotide analogues in the reaction mixture includes one or more predefined nucleoside analogue triphosphates selected from the group consisting of 2-Amino-ATP, 2-Amino-6-Cl-purine-rTP, 2′-F-dATP, 2′-Azido-dATP, 7-Deaza-ATP, 2-Cl-ATP, 2′-Bromo-dATP, 2-Fluoro-ATP, 8-Bromo-ATP, 8-Iodo-ATP, 2′-Iodo-dATP, 1-Thio-UTP, 1-Thio-GTP, 1-Thio-CTP, 1-Thio-ATP, 1-Thio-dTTP, 1-Thio-dGTP, 1-Thio-dCTP, 1-Thio-dATP, 5-(Cyanine 7)-UTP, Cyanine 5-UTP, 6-(Cyanine 5)-CTP, 6-(Cyanine 3)-UTP, 6-(Cyanine 3)-CTP, Cyanine 5-dUTP, Cyanine 5-dCTP, Cyanine 3-dUTP, Cyanine 3-dCTP, Biotin-16-7-Deaza-dGTP, Desthiobiotin-16-UTP, Desthiobiotin-6-dCTP, Dabcyl-5-3-Aminoallyl-2′-dUTP, Biotin-16-AA-UTP, N4-Biotin-dCTP, Biotin-16-AA-CTP, Biotin-16-AA-dCTP, Biotin-16-AA-dUTP, dTTP, dGTP, dCTP, dATP, 7-Deaza-7-Propargylamino-dGTP, 7-Deaza-7-Propargylamino-dATP, 5-Formyl-dUTP, 5-cadUTP, 5-Indolyl-AA-dUTP, 5-Formyl-dCTP, 5-Propargylamino-dUTP, 5-Propargylamino-dCTP, 5-Hydroxymethyl-dUTP, 2′-Deoxyzebularine-TP, N4-Methyl-dCTP, 5-AA-dUTP, 5-AA-dCTP, 8-Chloro-dATP, 6-Thio-dGTP, 6-Aza-dUTP, 2-Thio-dCTP, 4-Thio-dTTP, 5-Hydroxy-dCTP, dPTP, 2-Thio-dTTP, 8-Oxo-dGTP, 8-Oxo-dATP, 5-Nitro-1-Indolyl-drTP, N2-Methyl-dGTP, O6-Methyl-dGTP, 5-Methyl-dCTP, N6-Methyl-dATP, 5-Iodo-dUTP, 5-Iodo-dCTP, 5-Fluoro-dUTP, dUTP, 5-Propynyl-dUTP, 5-Propynyl-dCTP, dITP, 7-Deaza-dGTP, 6-Cl-purine-drTP, 5-Br-dUTP, 5-Br-dCTP, 2-Aminopurine-drTP, 2-Amino-dATP, 2-Amino-6-Cl-purine-drTP, N1-Propyl-Pseudo-UTP, N1-MOM-Pseudo-UTP, N1-Ethylpseudo-UTP, Ara-GTP, Iso-GTP, 8-Oxo-ATP, Thieno-CTP, 5-Carboxymethylester-UTP, Thieno-UTP, 5-Methoxy-CTP, 5-Methoxy-UTP, 5-Hydroxy-UTP, 5-Carboxy-UTP, 5-Formyl-UTP, 5-Hydroxy-CTP, Thieno-GTP, 5-Hydroxymethyl-CTP, 5-Hydroxymethyl-UTP, 5-Formyl-CTP, N6-Methyl-Amino-ATP, 5,6-Dihydro-5-Me-UTP, m1ΨTP, N4-Methyl-CTP, 2-Aminopurine-rTP, 8-Oxo-GTP, 5-AA-CTP, 5-AA-UTP, 5-Br-UTP, 5-Br-CTP, 8-Azido-ATP, 7-Deaza-GTP, N1-Methyl-ATP, 6-Aza-UTP, 6-Aza-CTP, 2-Thio-CTP, 5,6-Dihydro-UTP, 2-Thio-UTP, O6-Methyl-GTP, 4-Thio-UTP, 5-Methyl-UTP, Xanthosine-TP, ITP, 5-Iodo-UTP, 5-Iodo-CTP, 6-Cl-purine-rTP, 8-Aza-ATP, and combination thereof.

In some embodiments, provided is a method, wherein the one or more predefined nucleoside analogue triphosphates are selected from the group consisting of 2-Amino-ATP, 2-Amino-6-Cl-purine-rTP, 2′-F-dATP, 2′-Azido-dATP, and 7-Deaza-ATP, and combination thereof.

In some embodiments, the poly(A) polymerase is a wild type E. coli poly(A) polymerase, or a mutant thereof, or a homolog thereof.

In one further example, the plurality of adenine nucleotides are oligonucleotides, i.e. oligo adenine nucleotide.

In some embodiments, the second sequence includes at least 10 adenine nucleotides.

In some embodiments, the one or more predefined nucleoside analogue triphosphates are 2-Amino-6-Cl-purine-rTP, and step (i) further includes the following step: incubating the sample with the reaction mixture for a predetermined time of at least 5 minutes.

In some embodiments, the predetermined time is about 10-20 minutes.

In some embodiments, provided is a method for sample preparation for direct RNA sequencing, including the steps of: (i) reacting the sample including one or more RNA molecules with a reaction mixture including a poly(A) polymerase and one or more predefined nucleotide analogues to add a first sequence including one or more predefined nucleotide analogues to individual 3′-end of the one or more RNA molecules, such that one or more first tailed RNA is formed; and (ii) adding a plurality of adenosine triphosphates (ATP) to the reaction mixture, such that the one or more first tailed RNA is reacted with the plurality of adenosine triphosphates (ATP) to add a second sequence including a plurality of adenine nucleotides to individual 3′-end of the one or more first tailed RNA, such that one or more second tailed RNA is formed.

In some embodiments, the method further includes the following steps: iii) adding a sequencing adapter and a ligase to the reaction mixture, such that the sequencing adapter is ligated to the one or more second tailed RNA, such that one or more ligated RNA is formed; and iv) purifying the one or more ligated RNA from the reaction mixture.

In some embodiments, the sequencing adapter includes an oligo-(dT) overhang.

In some embodiments, the one or more predefined nucleotide analogues in the reaction mixture includes one or more predefined nucleoside analogue triphosphates selected from the group consisting of 2-Amino-ATP, 2-Amino-6-Cl-purine-rTP, 2′-F-dATP, 2′-Azido-dATP, 7-Deaza-ATP, 2-Cl-ATP, 2′-Bromo-dATP, 2-Fluoro-ATP, 8-Bromo-ATP, 8-Iodo-ATP, 2′-Iodo-dATP, 1-Thio-UTP, 1-Thio-GTP, 1-Thio-CTP, 1-Thio-ATP, 1-Thio-dTTP, 1-Thio-dGTP, 1-Thio-dCTP, 1-Thio-dATP, 5-(Cyanine 7)-UTP, Cyanine 5-UTP, 6-(Cyanine 5)-CTP, 6-(Cyanine 3)-UTP, 6-(Cyanine 3)-CTP, Cyanine 5-dUTP, Cyanine 5-dCTP, Cyanine 3-dUTP, Cyanine 3-dCTP, Biotin-16-7-Deaza-dGTP, Desthiobiotin-16-UTP, Desthiobiotin-6-dCTP, Dabcyl-5-3-Aminoallyl-2′-dUTP, Biotin-16-AA-UTP, N4-Biotin-dCTP, Biotin-16-AA-CTP, Biotin-16-AA-dCTP, Biotin-16-AA-dUTP, dTTP, dGTP, dCTP, dATP, 7-Deaza-7-Propargylamino-dGTP, 7-Deaza-7-Propargylamino-dATP, 5-Formyl-dUTP, 5-cadUTP, 5-Indolyl-AA-dUTP, 5-Formyl-dCTP, 5-Propargylamino-dUTP, 5-Propargylamino-dCTP, 5-Hydroxymethyl-dUTP, 2′-Deoxyzebularine-TP, N4-Methyl-dCTP, 5-AA-dUTP, 5-AA-dCTP, 8-Chloro-dATP, 6-Thio-dGTP, 6-Aza-dUTP, 2-Thio-dCTP, 4-Thio-dTTP, 5-Hydroxy-dCTP, dPTP, 2-Thio-dTTP, 8-Oxo-dGTP, 8-Oxo-dATP, 5-Nitro-1-Indolyl-drTP, N2-Methyl-dGTP, O6-Methyl-dGTP, 5-Methyl-dCTP, N6-Methyl-dATP, 5-Iodo-dUTP, 5-Iodo-dCTP, 5-Fluoro-dUTP, dUTP, 5-Propynyl-dUTP, 5-Propynyl-dCTP, dITP, 7-Deaza-dGTP, 6-Cl-purine-drTP, 5-Br-dUTP, 5-Br-dCTP, 2-Aminopurine-drTP, 2-Amino-dATP, 2-Amino-6-Cl-purine-drTP, N1-Propyl-Pseudo-UTP, N1-MOM-Pseudo-UTP, N1-Ethylpseudo-UTP, Ara-GTP, Iso-GTP, 8-Oxo-ATP, Thieno-CTP, 5-Carboxymethylester-UTP, Thieno-UTP, 5-Methoxy-CTP, 5-Methoxy-UTP, 5-Hydroxy-UTP, 5-Carboxy-UTP, 5-Formyl-UTP, 5-Hydroxy-CTP, Thieno-GTP, 5-Hydroxymethyl-CTP, 5-Hydroxymethyl-UTP, 5-Formyl-CTP, N6-Methyl-Amino-ATP, 5,6-Dihydro-5-Me-UTP, m1ΨTP, N4-Methyl-CTP, 2-Aminopurine-rTP, 8-Oxo-GTP, 5-AA-CTP, 5-AA-UTP, 5-Br-UTP, 5-Br-CTP, 8-Azido-ATP, 7-Deaza-GTP, N1-Methyl-ATP, 6-Aza-UTP, 6-Aza-CTP, 2-Thio-CTP, 5,6-Dihydro-UTP, 2-Thio-UTP, O6-Methyl-GTP, 4-Thio-UTP, 5-Methyl-UTP, Xanthosine-TP, ITP, 5-Iodo-UTP, 5-Iodo-CTP, 6-Cl-purine-rTP, 8-Aza-ATP, and combination thereof.

In some embodiments, provided is a method, wherein the one or more predefined nucleoside analogue triphosphates are selected from the group consisting of 2-Amino-ATP, 2-Amino-6-Cl-purine-rTP, 2′-F-dATP, 2′-Azido-dATP, and 7-Deaza-ATP, and combination thereof.

In some embodiments, the poly(A) polymerase is a wild type E. coli poly(A) polymerase, or a mutant thereof, or a homolog thereof.

In some embodiments, the second sequence includes at least 10 adenine nucleotides.

In some embodiments, the one or more predefined nucleoside analogue triphosphates are 2-Amino-6-Cl-purine-rTP, and step (i) further includes the following step: incubating the sample with the reaction mixture for a predetermined time of at least 5 minutes.

In some embodiments, the predetermined time is about 10-20 minutes.

In some embodiments, provided is a kit for preparing a sample including one or more RNA molecules for sequencing, including: (a) a poly(A) polymerase; (b) one or more predefined nucleotide analogues; and (c) a plurality of adenosine triphosphates (ATP), and optionally, d) a ligase; e) a sequencing adapter; and/or f) one or more buffer solutions.

In some embodiments, the kit further includes: instructions for preparing the sample including one or more RNA molecules by the following steps: (i) reacting the sample with a reaction mixture including the poly(A) polymerase and the one or more predefined nucleotide analogues to add a first sequence including one or more predefined nucleotide analogues to individual 3′-end of the one or more RNA molecules, such that one or more first tailed RNA is formed; and (ii) adding a plurality of adenosine triphosphates (ATP) to the reaction mixture, such that the one or more first tailed RNA is reacted with the plurality of adenosine triphosphates (ATP) to add a second sequence including a plurality of adenine nucleotides to individual 3′-end of the one or more first tailed RNA, such that one or more second tailed RNA is formed.

In some embodiments, the one or more predefined nucleotide analogues includes one or more nucleoside analogue triphosphates selected from the group consisting of 2-Amino-ATP, 2-Amino-6-Cl-purine-rTP, 2′-F-dATP, 2′-Azido-dATP, 7-Deaza-ATP, 2-Cl-ATP, 2′-Bromo-dATP, 2-Fluoro-ATP, 8-Bromo-ATP, 8-Iodo-ATP, 2′-Iodo-dATP, 1-Thio-UTP, 1-Thio-GTP, 1-Thio-CTP, 1-Thio-ATP, 1-Thio-dTTP, 1-Thio-dGTP, 1-Thio-dCTP, 1-Thio-dATP, 5-(Cyanine 7)-UTP, Cyanine 5-UTP, 6-(Cyanine 5)-CTP, 6-(Cyanine 3)-UTP, 6-(Cyanine 3)-CTP, Cyanine 5-dUTP, Cyanine 5-dCTP, Cyanine 3-dUTP, Cyanine 3-dCTP, Biotin-16-7-Deaza-dGTP, Desthiobiotin-16-UTP, Desthiobiotin-6-dCTP, Dabcyl-5-3-Aminoallyl-2′-dUTP, Biotin-16-AA-UTP, N4-Biotin-dCTP, Biotin-16-AA-CTP, Biotin-16-AA-dCTP, Biotin-16-AA-dUTP, dTTP, dGTP, dCTP, dATP, 7-Deaza-7-Propargylamino-dGTP, 7-Deaza-7-Propargylamino-dATP, 5-Formyl-dUTP, 5-cadUTP, 5-Indolyl-AA-dUTP, 5-Formyl-dCTP, 5-Propargylamino-dUTP, 5-Propargylamino-dCTP, 5-Hydroxymethyl-dUTP, 2′-Deoxyzebularine-TP, N4-Methyl-dCTP, 5-AA-dUTP, 5-AA-dCTP, 8-Chloro-dATP, 6-Thio-dGTP, 6-Aza-dUTP, 2-Thio-dCTP, 4-Thio-dTTP, 5-Hydroxy-dCTP, dPTP, 2-Thio-dTTP, 8-Oxo-dGTP, 8-Oxo-dATP, 5-Nitro-1-Indolyl-drTP, N2-Methyl-dGTP, O6-Methyl-dGTP, 5-Methyl-dCTP, N6-Methyl-dATP, 5-Iodo-dUTP, 5-Iodo-dCTP, 5-Fluoro-dUTP, dUTP, 5-Propynyl-dUTP, 5-Propynyl-dCTP, dITP, 7-Deaza-dGTP, 6-Cl-purine-drTP, 5-Br-dUTP, 5-Br-dCTP, 2-Aminopurine-drTP, 2-Amino-dATP, 2-Amino-6-Cl-purine-drTP, N1-Propyl-Pseudo-UTP, N1-MOM-Pseudo-UTP, N1-Ethylpseudo-UTP, Ara-GTP, Iso-GTP, 8-Oxo-ATP, Thieno-CTP, 5-Carboxymethylester-UTP, Thieno-UTP, 5-Methoxy-CTP, 5-Methoxy-UTP, 5-Hydroxy-UTP, 5-Carboxy-UTP, 5-Formyl-UTP, 5-Hydroxy-CTP, Thieno-GTP, 5-Hydroxymethyl-CTP, 5-Hydroxymethyl-UTP, 5-Formyl-CTP, N6-Methyl-Amino-ATP, 5,6-Dihydro-5-Me-UTP, m1ΨTP, N4-Methyl-CTP, 2-Aminopurine-rTP, 8-Oxo-GTP, 5-AA-CTP, 5-AA-UTP, 5-Br-UTP, 5-Br-CTP, 8-Azido-ATP, 7-Deaza-GTP, N1-Methyl-ATP, 6-Aza-UTP, 6-Aza-CTP, 2-Thio-CTP, 5,6-Dihydro-UTP, 2-Thio-UTP, O6-Methyl-GTP, 4-Thio-UTP, 5-Methyl-UTP, Xanthosine-TP, ITP, 5-Iodo-UTP, 5-Iodo-CTP, 6-Cl-purine-rTP, 8-Aza-ATP, and combination thereof.

In some embodiments, the one or more predefined nucleoside analogue triphosphates are selected from the group consisting of 2-Amino-ATP, 2-Amino-6-Cl-purine-rTP, 2′-F-dATP, 2′-Azido-dATP, and 7-Deaza-ATP, and combination thereof.

Although the description referred to particular embodiments, the disclosure should not be construed as limited to the embodiments set forth herein.

### NUMBERED EMBODIMENTS

Embodiment 1. A method for preparing a sample comprising one or more RNA molecules, comprising the steps of: (i) reacting the sample with a reaction mixture comprising a poly(A) polymerase and one or more predefined nucleotide analogues to add a first sequence comprising one or more predefined nucleotide analogues to individual 3′-end of the one or more RNA molecules, such that one or more first tailed RNA is formed; and (ii) adding a plurality of adenosine triphosphates (ATP) to the reaction mixture, such that the one or more first tailed RNA is reacted with the plurality of adenosine triphosphates (ATP) to add a second sequence comprising a plurality of adenine nucleotides to individual 3′-end of the one or more first tailed RNA, such that one or more second tailed RNA is formed.

Embodiment 2. The method of embodiment 1, wherein the one or more predefined nucleotide analogues in the reaction mixture comprises one or more predefined nucleoside analogue triphosphates selected from the group consisting of 2-Amino-ATP, 2-Amino-6-Cl-purine-rTP, 2′-F-dATP, 2′-Azido-dATP, 7-Deaza-ATP, 2-CI-ATP, 2′-Bromo-dATP, 2-Fluoro-ATP, 8-Bromo-ATP, 8-Iodo-ATP, 2′-Iodo-dATP, 1-Thio-UTP, 1-Thio-GTP, 1-Thio-CTP, 1-Thio-ATP, 1-Thio-dTTP, 1-Thio-dGTP, 1-Thio-dCTP, 1-Thio-dATP, 5-(Cyanine 7)-UTP, Cyanine 5-UTP, 6-(Cyanine 5)-CTP, 6-(Cyanine 3)-UTP, 6-(Cyanine 3)-CTP, Cyanine 5-dUTP, Cyanine 5-dCTP, Cyanine 3-dUTP, Cyanine 3-dCTP, Biotin-16-7-Deaza-dGTP, Desthiobiotin-16-UTP, Desthiobiotin-6-dCTP, Dabcyl-5-3-Aminoallyl-2′-dUTP, Biotin-16-AA-UTP, N4-Biotin-dCTP, Biotin-16-AA-CTP, Biotin-16-AA-dCTP, Biotin-16-AA-dUTP, dTTP, dGTP, dCTP, dATP, 7-Deaza-7-Propargylamino-dGTP, 7-Deaza-7-Propargylamino-dATP, 5-Formyl-dUTP, 5-cadUTP, 5-Indolyl-AA-dUTP, 5-Formyl-dCTP, 5-Propargylamino-dUTP, 5-Propargylamino-dCTP, 5-Hydroxymethyl-dUTP, 2′-Deoxyzebularine-TP, N4-Methyl-dCTP, 5-AA-dUTP, 5-AA-dCTP, 8-Chloro-dATP, 6-Thio-dGTP, 6-Aza-dUTP, 2-Thio-dCTP, 4-Thio-dTTP, 5-Hydroxy-dCTP, dPTP, 2-Thio-dTTP, 8-Oxo-dGTP, 8-Oxo-dATP, 5-Nitro-1-Indolyl-drTP, N2-Methyl-dGTP, O6-Methyl-dGTP, 5-Methyl-dCTP, N6-Methyl-dATP, 5-Iodo-dUTP, 5-Iodo-dCTP, 5-Fluoro-dUTP, dUTP, 5-Propynyl-dUTP, 5-Propynyl-dCTP, dITP, 7-Deaza-dGTP, 6-Cl-purine-drTP, 5-Br-dUTP, 5-Br-dCTP, 2-Aminopurine-drTP, 2-Amino-dATP, 2-Amino-6-Cl-purine-drTP, N1-Propyl-Pseudo-UTP, N1-MOM-Pseudo-UTP, N1-Ethylpseudo-UTP, Ara-GTP, Iso-GTP, 8-Oxo-ATP, Thieno-CTP, 5-Carboxymethylester-UTP, Thieno-UTP, 5-Methoxy-CTP, 5-Methoxy-UTP, 5-Hydroxy-UTP, 5-Carboxy-UTP, 5-Formyl-UTP, 5-Hydroxy-CTP, Thieno-GTP, 5-Hydroxymethyl-CTP, 5-Hydroxymethyl-UTP, 5-Formyl-CTP, N6-Methyl-Amino-ATP, 5,6-Dihydro-5-Me-UTP, m1ΨTP, N4-Methyl-CTP, 2-Aminopurine-rTP, 8-Oxo-GTP, 5-AA-CTP, 5-AA-UTP, 5-Br-UTP, 5-Br-CTP, 8-Azido-ATP, 7-Deaza-GTP, N1-Methyl-ATP, 6-Aza-UTP, 6-Aza-CTP, 2-Thio-CTP, 5,6-Dihydro-UTP, 2-Thio-UTP, O6-Methyl-GTP, 4-Thio-UTP, 5-Methyl-UTP, Xanthosine-TP, ITP, 5-Iodo-UTP, 5-Iodo-CTP, 6-Cl-purine-rTP, 8-Aza-ATP, and combination thereof.

Embodiment 3. The method of embodiment 2, wherein the one or more predefined nucleoside analogue triphosphates are selected from the group consisting of 2-Amino-ATP, 2-Amino-6-Cl-purine-rTP, 2′-F-dATP, 2′-Azido-dATP, and 7-Deaza-ATP, and combination thereof.

Embodiment 4. The method of embodiment 1, wherein the poly(A) polymerase is a wild type E. coli poly(A) polymerase, or a mutant thereof, or a homolog thereof.

Embodiment 5. The method of embodiment 1, wherein the second sequence comprises at least 10 adenine nucleotides.

Embodiment 6. The method of embodiment 2, wherein the one or more predefined nucleoside analogue triphosphates are 2-Amino-6-Cl-purine-rTP, and step (i) further includes the following step: incubating the sample with the reaction mixture for a predetermined time of at least 5 minutes.

Embodiment 7. The method of embodiment 6, wherein the predetermined time is about 10-20 minutes.

Embodiment 8. A method for sample preparation for direct RNA sequencing, comprising the steps of: (i) reacting the sample comprising one or more RNA molecules with a reaction mixture comprising a poly(A) polymerase and one or more predefined nucleotide analogues to add a first sequence comprising one or more predefined nucleotide analogues to individual 3′-end of the one or more RNA molecules, such that one or more first tailed RNA is formed; and (ii) adding a plurality of adenosine triphosphates (ATP) to the reaction mixture, such that the one or more first tailed RNA is reacted with the plurality of adenosine triphosphates (ATP) to add a second sequence comprising a plurality of adenine nucleotides to individual 3′-end of the one or more first tailed RNA, such that one or more second tailed RNA is formed.

Embodiment 9. The method of embodiment 8, wherein the method further comprises the following steps: iii) adding a sequencing adapter and a ligase to the reaction mixture, such that the sequencing adapter is ligated to the one or more second tailed RNA, such that one or more ligated RNA is formed; and iv) purifying the one or more ligated RNA from the reaction mixture.

Embodiment 10. The method of embodiment 9, wherein the sequencing adapter comprises an oligo-(dT) overhang.

Embodiment 11. The method of embodiment 8, wherein the one or more predefined nucleotide analogues in the reaction mixture comprises one or more predefined nucleoside analogue triphosphates selected from the group consisting of 2-Amino-ATP, 2-Amino-6-Cl-purine-rTP, 2′-F-dATP, 2′-Azido-dATP, 7-Deaza-ATP, 2-Cl-ATP, 2′-Bromo-dATP, 2-Fluoro-ATP, 8-Bromo-ATP, 8-Iodo-ATP, 2′-Iodo-dATP, 1-Thio-UTP, 1-Thio-GTP, 1-Thio-CTP, 1-Thio-ATP, 1-Thio-dTTP, 1-Thio-dGTP, 1-Thio-dCTP, 1-Thio-dATP, 5-(Cyanine 7)-UTP, Cyanine 5-UTP, 6-(Cyanine 5)-CTP, 6-(Cyanine 3)-UTP, 6-(Cyanine 3)-CTP, Cyanine 5-dUTP, Cyanine 5-dCTP, Cyanine 3-dUTP, Cyanine 3-dCTP, Biotin-16-7-Deaza-dGTP, Desthiobiotin-16-UTP, Desthiobiotin-6-dCTP, Dabcyl-5-3-Aminoallyl-2′-dUTP, Biotin-16-AA-UTP, N4-Biotin-dCTP, Biotin-16-AA-CTP, Biotin-16-AA-dCTP, Biotin-16-AA-dUTP, dTTP, dGTP, dCTP, dATP, 7-Deaza-7-Propargylamino-dGTP, 7-Deaza-7-Propargylamino-dATP, 5-Formyl-dUTP, 5-cadUTP, 5-Indolyl-AA-dUTP, 5-Formyl-dCTP, 5-Propargylamino-dUTP, 5-Propargylamino-dCTP, 5-Hydroxymethyl-dUTP, 2′-Deoxyzebularine-TP, N4-Methyl-dCTP, 5-AA-dUTP, 5-AA-dCTP, 8-Chloro-dATP, 6-Thio-dGTP, 6-Aza-dUTP, 2-Thio-dCTP, 4-Thio-dTTP, 5-Hydroxy-dCTP, dPTP, 2-Thio-dTTP, 8-Oxo-dGTP, 8-Oxo-dATP, 5-Nitro-1-Indolyl-drTP, N2-Methyl-dGTP, O6-Methyl-dGTP, 5-Methyl-dCTP, N6-Methyl-dATP, 5-Iodo-dUTP, 5-Iodo-dCTP, 5-Fluoro-dUTP, dUTP, 5-Propynyl-dUTP, 5-Propynyl-dCTP, dITP, 7-Deaza-dGTP, 6-Cl-purine-drTP, 5-Br-dUTP, 5-Br-dCTP, 2-Aminopurine-drTP, 2-Amino-dATP, 2-Amino-6-Cl-purine-drTP, N1-Propyl-Pseudo-UTP, N1-MOM-Pseudo-UTP, N1-Ethylpseudo-UTP, Ara-GTP, Iso-GTP, 8-Oxo-ATP, Thieno-CTP, 5-Carboxymethylester-UTP, Thieno-UTP, 5-Methoxy-CTP, 5-Methoxy-UTP, 5-Hydroxy-UTP, 5-Carboxy-UTP, 5-Formyl-UTP, 5-Hydroxy-CTP, Thieno-GTP, 5-Hydroxymethyl-CTP, 5-Hydroxymethyl-UTP, 5-Formyl-CTP, N6-Methyl-Amino-ATP, 5,6-Dihydro-5-Me-UTP, m1ΨTP, N4-Methyl-CTP, 2-Aminopurine-rTP, 8-Oxo-GTP, 5-AA-CTP, 5-AA-UTP, 5-Br-UTP, 5-Br-CTP, 8-Azido-ATP, 7-Deaza-GTP, N1-Methyl-ATP, 6-Aza-UTP, 6-Aza-CTP, 2-Thio-CTP, 5,6-Dihydro-UTP, 2-Thio-UTP, O6-Methyl-GTP, 4-Thio-UTP, 5-Methyl-UTP, Xanthosine-TP, ITP, 5-Iodo-UTP, 5-Iodo-CTP, 6-Cl-purine-rTP, 8-Aza-ATP, and combination thereof.

Embodiment 12. The method of embodiment 11, wherein the one or more predefined nucleoside analogue triphosphates are selected from the group consisting of 2-Amino-ATP, 2-Amino-6-Cl-purine-rTP, 2′-F-dATP, 2′-Azido-dATP, and 7-Deaza-ATP, and combination thereof.

Embodiment 13. The method of embodiment 8, wherein the poly(A) polymerase is a wild type E. coli poly(A) polymerase, or a mutant thereof, or a homolog thereof.

Embodiment 14. The method of embodiment 8, wherein the second sequence comprises at least 10 adenine nucleotides.

Embodiment 15. The method of embodiment 8, wherein the one or more predefined nucleoside analogue triphosphates are 2-Amino-6-Cl-purine-rTP, and step (i) further includes the following step: incubating the sample with the reaction mixture for a predetermined time of at least 5 minutes.

Embodiment 16. The method of embodiment 15, wherein the predetermined time is about 10-20 minutes.

Embodiment 17. A kit for preparing a sample comprising one or more RNA molecules for sequencing, comprising: (a) a poly(A) polymerase; (b) one or more predefined nucleotide analogues; and (c) a plurality of adenosine triphosphates (ATP), and optionally, d) a ligase; e) a sequencing adapter; and/or f) one or more buffer solutions.

Embodiment 18. The kit of embodiment 17, further comprises: instructions for preparing the sample comprising one or more RNA molecules by the following steps: (i) reacting the sample with a reaction mixture comprising the poly(A) polymerase and the one or more predefined nucleotide analogues to add a first sequence comprising one or more predefined nucleotide analogues to individual 3′-end of the one or more RNA molecules, such that one or more first tailed RNA is formed; and (ii) adding a plurality of adenosine triphosphates (ATP) to the reaction mixture, such that the one or more first tailed RNA is reacted with the plurality of adenosine triphosphates (ATP) to add a second sequence comprising a plurality of adenine nucleotides to individual 3′-end of the one or more first tailed RNA, such that one or more second tailed RNA is formed.

Embodiment 19. The kit of embodiment 17, wherein the one or more predefined nucleotide analogues comprises one or more nucleoside analogue triphosphates selected from the group consisting of 2-Amino-ATP, 2-Amino-6-Cl-purine-rTP, 2′-F-dATP, 2′-Azido-dATP, 7-Deaza-ATP, 2-Cl-ATP, 2′-Bromo-dATP, 2-Fluoro-ATP, 8-Bromo-ATP, 8-Iodo-ATP, 2′-Iodo-dATP, 1-Thio-UTP, 1-Thio-GTP, 1-Thio-CTP, 1-Thio-ATP, 1-Thio-dTTP, 1-Thio-dGTP, 1-Thio-dCTP, 1-Thio-dATP, 5-(Cyanine 7)-UTP, Cyanine 5-UTP, 6-(Cyanine 5)-CTP, 6-(Cyanine 3)-UTP, 6-(Cyanine 3)-CTP, Cyanine 5-dUTP, Cyanine 5-dCTP, Cyanine 3-dUTP, Cyanine 3-dCTP, Biotin-16-7-Deaza-dGTP, Desthiobiotin-16-UTP, Desthiobiotin-6-dCTP, Dabcyl-5-3-Aminoallyl-2′-dUTP, Biotin-16-AA-UTP, N4-Biotin-dCTP, Biotin-16-AA-CTP, Biotin-16-AA-dCTP, Biotin-16-AA-dUTP, dTTP, dGTP, dCTP, dATP, 7-Deaza-7-Propargylamino-dGTP, 7-Deaza-7-Propargylamino-dATP, 5-Formyl-dUTP, 5-cadUTP, 5-Indolyl-AA-dUTP, 5-Formyl-dCTP, 5-Propargylamino-dUTP, 5-Propargylamino-dCTP, 5-Hydroxymethyl-dUTP, 2′-Deoxyzebularine-TP, N4-Methyl-dCTP, 5-AA-dUTP, 5-AA-dCTP, 8-Chloro-dATP, 6-Thio-dGTP, 6-Aza-dUTP, 2-Thio-dCTP, 4-Thio-dTTP, 5-Hydroxy-dCTP, dPTP, 2-Thio-dTTP, 8-Oxo-dGTP, 8-Oxo-dATP, 5-Nitro-1-Indolyl-drTP, N2-Methyl-dGTP, O6-Methyl-dGTP, 5-Methyl-dCTP, N6-Methyl-dATP, 5-Iodo-dUTP, 5-Iodo-dCTP, 5-Fluoro-dUTP, dUTP, 5-Propynyl-dUTP, 5-Propynyl-dCTP, dITP, 7-Deaza-dGTP, 6-Cl-purine-drTP, 5-Br-dUTP, 5-Br-dCTP, 2-Aminopurine-drTP, 2-Amino-dATP, 2-Amino-6-Cl-purine-drTP, N1-Propyl-Pseudo-UTP, N1-MOM-Pseudo-UTP, N1-Ethylpseudo-UTP, Ara-GTP, Iso-GTP, 8-Oxo-ATP, Thieno-CTP, 5-Carboxymethylester-UTP, Thieno-UTP, 5-Methoxy-CTP, 5-Methoxy-UTP, 5-Hydroxy-UTP, 5-Carboxy-UTP, 5-Formyl-UTP, 5-Hydroxy-CTP, Thieno-GTP, 5-Hydroxymethyl-CTP, 5-Hydroxymethyl-UTP, 5-Formyl-CTP, N6-Methyl-Amino-ATP, 5,6-Dihydro-5-Me-UTP, m1ΨTP, N4-Methyl-CTP, 2-Aminopurine-rTP, 8-Oxo-GTP, 5-AA-CTP, 5-AA-UTP, 5-Br-UTP, 5-Br-CTP, 8-Azido-ATP, 7-Deaza-GTP, N1-Methyl-ATP, 6-Aza-UTP, 6-Aza-CTP, 2-Thio-CTP, 5,6-Dihydro-UTP, 2-Thio-UTP, O6-Methyl-GTP, 4-Thio-UTP, 5-Methyl-UTP, Xanthosine-TP, ITP, 5-Iodo-UTP, 5-Iodo-CTP, 6-Cl-purine-rTP, 8-Aza-ATP, and combination thereof.

Embodiment 20. The kit of embodiment 19, wherein the one or more predefined nucleoside analogue triphosphates are selected from the group consisting of 2-Amino-ATP, 2-Amino-6-Cl-purine-rTP, 2′-F-dATP, 2′-Azido-dATP, and 7-Deaza-ATP, and combination thereof.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Example 1: Methods for Preparing a Sample Comprising One or More RNA Molecules for Sequencing (One Pot 3′ Extension)

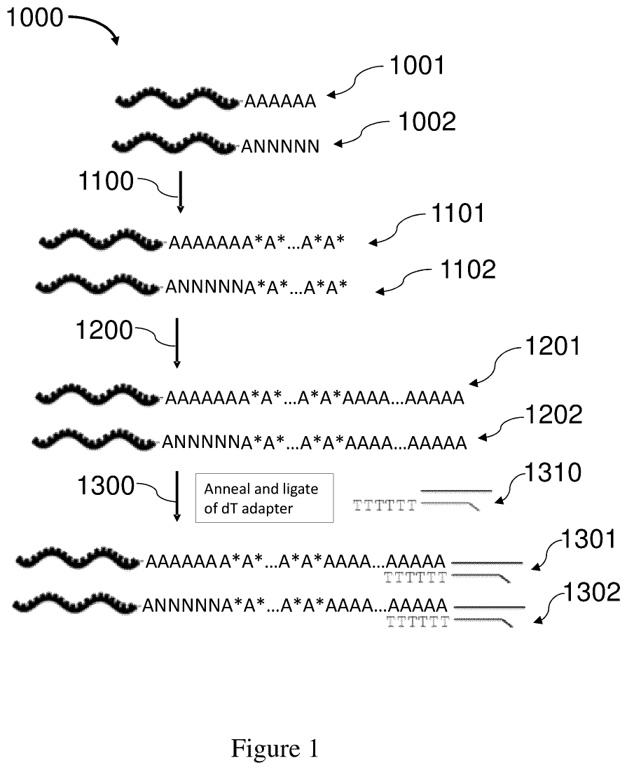

Referring now to FIG. 1, which shows a workflow of an example method 1000 for preparing a sample comprising one or more RNA molecules according to an example embodiment. For case of description, the example method is also referred to as “one-pot 3′ extension” herein.

An initial sample comprising a plurality of RNA molecules is provided. In this example, the RNA molecules are or contains messenger RNA (mRNA) molecules. FIG. 1 shows two example RNA molecules of the initial sample carrying different 3′ tail sequences, which includes a first RNA 1001 with 3′ termini containing A bases (illustrated as “AAAAAA” in FIG. 1) and a second RNA 1002 with 3′ termini containing non-A bases (illustrated as “ANNNNN” in FIG. 1, with “N” representing a non-A base).

In step 1100, the initial sample is reacted with a reaction mixture comprising a poly(A) polymerase and a plurality of predefined nucleotide analogues (each predefined nucleotide analogue is represented as “A*” in FIG. 1). The poly(A) polymerase is configured to add one or more predefined nucleotide analogues so as to add or introduce a first sequence with a contiguous nucleotide residues of A*(also referred to as “oligo A* sequence” or “oligo nucleotide analogue sequence”) to the respective 3′ termini of the first RNA 1001 and second RNA 1002, such that a first tailed RNA 1101 and a first tailed RNA 1102 are formed, respectively. In some embodiments, the reaction in step 1100 is also referred to as “poly(A) polymerase-based extension”.

In step 1200, a plurality of adenosine triphosphates (ATP) is added or introduced to the reaction mixture, such that the first tailed RNA 1101 and the first tailed RNA 1102 are reacted with the ATPs by the poly(A) polymerase, which is provided in step 1100. The poly(A) polymerase is configured to add a plurality of adenine nucleotides so as to add or introduce a second sequence with a contiguous nucleotide residues of A (also referred to as “oligo(A) sequence”) to the respective 3′ termini of the first tailed RNA 1101 and the first tailed RNA 1102, such that a second tailed RNA 1201 and a second tailed RNA 1202 are formed, respectively. As such, substantially all second tailed RNA molecules contain oligo(A) sequences for the subsequent RNA sequencing steps.

In this embodiment, step 1100 and step 1200 above are performed sequentially in the same reaction mixture with the poly(A) polymerase. In step 1200, the plurality of adenosine triphosphates (ATP) is added directly in the reaction mixture which includes the first tailed RNA 1101 and the first tailed RNA 1102 of step 1100, without the need to purify the reaction mixture prior to step 1200. The resulting mixture is incubated at the same reaction condition as step 1100. This makes the preparation steps easy to operate, while reducing the risk of RNA degradation by minimizing the processing steps. In this embodiment, the method does not comprise the step of purifying the one or more first tailed RNA prior to step 1200.

In step 1300, a sequencing adapter 1310 and a ligase are added to the reaction mixture, such that the sequencing adapter 1310 is ligated to the respective 3′ termini of the second tailed RNA 1201 and the second tailed RNA 1202. In this example, the sequencing adapter 1310 includes an oligo-(dT) overhang, which anneals to the respective oligo(A) sequences of the second tailed RNA 1201 and the second tailed RNA 1202, such that a first ligated RNA 1301 and a second ligated RNA 1302 are formed, respectively. As such, RNA molecules carrying different 3′ tail sequences can be unbiasedly captured by the sequencing adapter 1310 after the one-pot 3′ extension method 1000. In an optional step (not shown), the ligated RNAs are purified from the reaction mixture which may contain residual nucleotide analogues, ATPs, poly (A) polymerase, ligase, and/or sequencing adapter. The purified ligated RNAs can then be used for the subsequent RNA sequencing, for example Nanopore direct RNA sequencing. Because the nucleotide analogue (A*) is chemically different from natural nucleotides (A), the nanopore can distinguish A* to accurately locate the last base of the 3′ tail of each of the sequenced RNA molecules. As such, in some embodiments, the one-pot 3′ extension method enhances RNA ligation yield and maintain 3′ tail sequence integrity for direct RNA sequencing. In this embodiment, the sequencing adapter 1310 and the ligase are added directly in the reaction mixture which includes the second tailed RNA 1201 and the second tailed RNA 1202 of step 1200, without the need to purify the reaction mixture prior to step 1200. The method does not comprise the step of purifying the one or more second tailed RNA prior to step 1300.

### Example 2: Methods for Model RNAs Synthesis

Unless otherwise stated, different model RNAs as shown in Table 1 were used in the subsequent examples as described below. The model RNAs were prepared by the following example method. Firstly, the corresponding DNA templates were synthesized using PCR (Q5® High-Fidelity 2× Master Mix) to produce PCR products. The purified PCR products were directly used for in vitro synthesis of model RNAs using MEGAscript™ T7 Transcription Kit (Invitrogen) following the manufacturer's instructions. The model RNAs were synthesized with adenosine triphosphate (ATP), cytidine triphosphate (CTP), uridine triphosphate (UTP), guanosine triphosphate (GTP), and Anti Reverse Cap Analog (TriLink BioTechnologies) with a molar ratio at 5:5:5:1:4. The reaction product was purified using the Monarch® RNA cleanup kit (New England BioLabs®) after enzymatic treatments with Turbo™ DNase (Invitrogen™) and Antarctic phosphatase (New England BioLabs®) following the manufacturers' protocol.

TABLE 1
List of the model RNAs

| | | | Length in | |
|---|---|---|---|---|
| | | SEQ ID | nucleotides | |
| Name | Tail sequence | NO: | (nt) | Remark |
| Model short RNA | AAAAAAAAAAAAAAAAAA | SEQ ID | 136 nt | |
| (A-terminated) | AAAAAAAAAAAAAAAAAA | NO: 1 | | |
| | AAAA | | | |
| Model short RNA | AAAAAAAAAAAAAAAAAA | SEQ ID | 136 nt | |
| (C-terminated) | AAAAAAAAAAAAAAAAAA | NO: 2 | | |
| | AAAC | | | |
| Model short RNA | AAAAAAAAAAAAAAAAAA | SEQ ID | 136 nt | |
| (G-terminated) | AAAAAAAAAAAAAAAAAA | NO: 3 | | |
| | AAAG | | | |
| Model short RNA | AAAAAAAAAAAAAAAAAA | SEQ ID | 136 nt | “U” refers |
| (U-terminated) | AAAAAAAAAAAAAAAAAA | NO: 4 | | to Uracil |
| | AAAU | | | |
| CLuc-53AGGCCCCU | AAAAAAAAAAAAAAAAAA | SEQ ID | 1871 nt | Used in the |
| | AAAAAAAAAAAAAAAAAA | NO: 5 | | model RNA |
| | AAAAAAAAAAAAAAAAAG | | | mixture; |
| | GCCCCU | | | “U” refers |
| | | | | to Uracil |
| EGFP-40A | AAAAAAAAAAAAAAAAAA | SEQ ID | 936 nt | Used in the |
| | AAAAAAAAAAAAAAAAAA | NO: 6 | | model RNA |
| | AAAA | | | mixture |
| UnaG-120A | AAAAAAAAAAAAAAAAAA | SEQ ID | 689 nt | Used in the |
| | AAAAAAAAAAAAAAAAAA | NO: 7 | | model RNA |
| | AAAAAAAAAAAAAAAAAA | | | mixture |
| | AAAAAAAAAAAAAAAAAA | | | |
| | AAAAAAAAAAAAAAAAAA | | | |
| | AAAAAAAAAAAAAAAAAA | | | |
| | AAAAAAAAAAAA | | | |

### Example 3: Reaction Cocktail for Poly(A) Polymerase-Based Extension

Table 2 shows the reaction cocktail for an example poly(A) polymerase-based extension. The reaction cocktail was used in the subsequent studies as described in examples below.

TABLE 2
Poly(A) polymerase-based extension reaction cocktail

| | | Final | |
|---|---|---|---|
| | Volume | concentration/ | |
| Component | (μL) | amount | |
| RNA sample | | 100 ng of | |
| | | total RNA | |
| Poly(A) polymerase reaction | 1 | 1x | |
| buffer (10x) (B0276SVIAL, | | | |
| New England BioLabs ®) | | | |
| ATP or nucleotide analogues | 1 | 1 | mM |
| (triphosphate forms) (10 mM) | | | |
| (Trilink Biotechnologies) | | | |
| E coli. poly(A) polymerase | 0.5 | 2.5 | units |

| | |
|---|---|
| (5000 units/mL, M0276SVIAL, | |
| New England BioLabs ®) | |
| Water | Top up to the |
| | final volume |
| | of 10 μL |

### Example 4: Addition of Oligo Nucleotide Analogue Sequence to the 3′ Termini of RNAs by Poly(A) Polymerase-Based Extension

### Materials and Methods

In this example, 5 nucleoside analogue triphosphates (Trilink BioTechnologies) as shown in Table 3, i.e. 2-Amino-ATP, 2-Amino-6-Cl-purine-rTP, 2′-F-dATP, 2′-Azido-dATP and 7-Deaza-ATP, have been tested for poly(A) polymerase-based extension to generate the respective oligo nucleotide analogue sequence to the 3′ termini of model short RNA (A-terminated) as shown in Table 1. ATP was used as the positive control. The reaction cocktail for the poly(A) polymerase-based extension for each of the nucleoside analogue triphosphates and ATP was prepared as described in Table 2. The 10 μL of the reaction cocktail was incubated at 37° C. for 15 minutes to allow the extension of the adenine nucleotides (A) or nucleotide analogues. The reacted RNA products were visualized using Urea-PAGE.

The experiment was further repeated for 2-Amino-6-Cl-purine-rTP addition by poly(A) polymerase-based extension with different incubation times, i.e. 5, 10, 15, 20 minutes respectively. Addition of ATP by poly(A) polymerase-based extension with 20-minute incubation time was used as the positive control. The reacted RNA products were visualized using Urea-PAGE.

TABLE 3
List of the nucleotide candidates (in nucleoside triphosphates form) used in the reactions

| Name | Structure (As shown in the manufacturers' product page) |
|---|---|
| ATP | 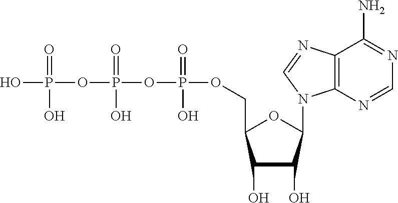 |
| 2-Amino-ATP | 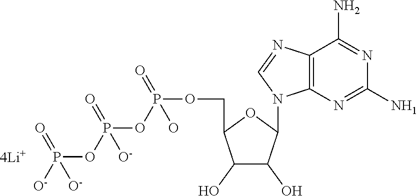 [SMILES: Nc1nc(N)c2ncn(C3OC(COP(=O)(O)OP(=O)([O-])OP(=O)([O-])[O-])C(O)C3O)c2n1] |
| 2-Amino-6-Cl-purine-rTP | 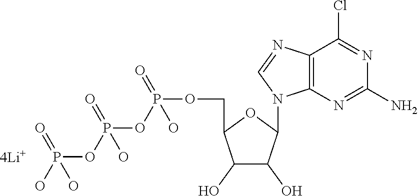 [SMILES: Nc1nc(Cl)c2ncn(C3OC(COP(=O)(O)OP(=O)(O)OP(=O)(O)O)C(O)C3O)c2n1] |
| 2′-F-dATP | 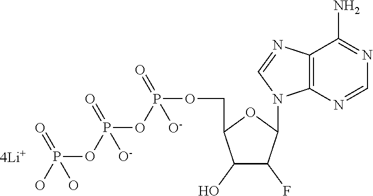 [SMILES: Nc1ncnc2c1ncn2C1OC(COP(=O)([O-])OP(=O)([O-])OP(=O)(O)O)C(O)C1F] |
| 2′-Azido-dATP | 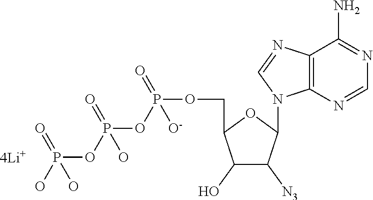 [SMILES: [N-]=[N+]=NC1C(O)C(COP(=O)([O-])OP(=O)(O)OP(=O)(O)O)OC1n1cnc2c(N)ncnc21] |
| 7-Deaza-ATP | 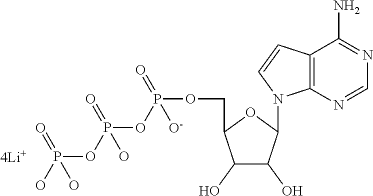 [SMILES: Nc1ncnc2c1ccn2C1OC(COP(=O)([O-])OP(=O)(O)OP(=O)(O)O)C(O)C1O] |

### Results

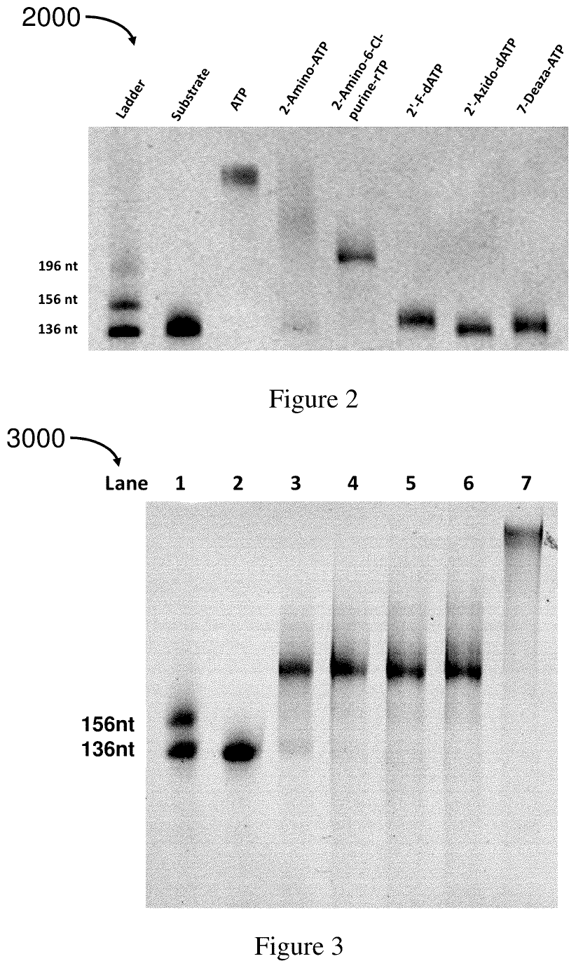

FIG. 2 shows the Urea-PAGE result 2000 of the reacted RNA products after the reactions of 2-Amino-ATP, 2-Amino-6-Cl-purine-rTP, 2′-F-dATP, 2′-Azido-dATP, 7-Deaza-ATP, and ATP (positive control) with the model short RNA respectively by poly(A) polymerase. The results showed that poly(A) polymerase-based extension at least successfully added 2-Amino-ATP, 2-Amino-6-Cl-purine-rTP, and 2′-F-dATP sequences to the 3′ termini of the model short RNA respectively.

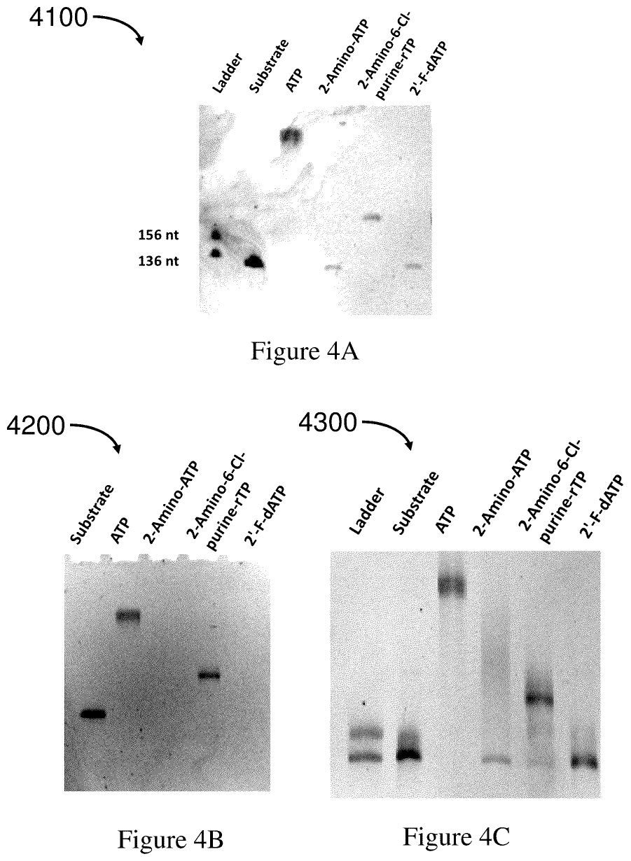

FIG. 3 shows the Urea-PAGE result 3000 of reacted RNA products after the reaction of 2-Amino-6-Cl-purine-rTP with the model short RNA by poly(A) polymerase with different incubation times. Lane 1 shows the 136 nt, 156 nt RNA ladder; Lane 2 shows the original model short RNA; Lane 3 shows the tailed RNA with 5-minute incubation time for adding 2-Amino-6-Cl-purine-rTP; Lane 4 shows the tailed RNA with 10-minute incubation time for adding 2-Amino-6-Cl-purine-rTP; Lane 5 shows the tailed RNA with 15-minute incubation time for adding 2-Amino-6-Cl-purine-rTP; Lane 6 shows the tailed RNA with 20-minute incubation time for adding 2-Amino-6-Cl-purine-rTP; Lane 7 shows the tailed RNA with 20-minute incubation time for adding ATP.

As shown in Lane 3, approximately over 60 nts of 2-Amino-6-Cl-purine-rTP were added to the 3′ terminus of model short RNA after incubating for as short as 5 minutes, forming a tailed model short RNA product longer than 196 nt. Further incubation for 10, 15 and 20 minutes, as shown in Lanes 4-6 respectively, will increase the yield of the reacted product but will not elongate the 2-Amino-6-Cl-purine-rTP sequence at the 3′ termini of the model short RNA. These results showed poly(A) polymerase-based extension successfully added oligo 2-Amino-6-Cl-purine-rTP to the 3′ terminus of RNA and the reaction was complete within 10-20 minutes.

### Example 5: Addition of Oligo Nucleotide Analogue Sequences to the 3′ Termini of the Model Short RNAs with Different 3′ Terminal Bases by Poly(A) Polymerase-Based Extension

### Materials and Methods

In this example, 2-Amino-ATP, 2-Amino-6-Cl-purine-rTP and 2′-F-dATP have been tested for poly(A) polymerase-based extension to add the respective oligo nucleotide analogue sequences to the 3′ termini of model short RNAs with different terminal bases other than A, i.e. C, G and U-terminated model short RNAs as shown in Table 1. ATP was used as the positive control. The reaction cocktail for the poly(A) polymerase-based extension for each model short RNA was prepared as described in Table 2. The 10 μL of the reaction mixture was incubated at 37° C. for 15 minutes to allow the addition of 2-Amino-6-Cl-purine-rTP to the model short RNAs. The reacted RNA products were visualized using Urea-PAGE.

### Results

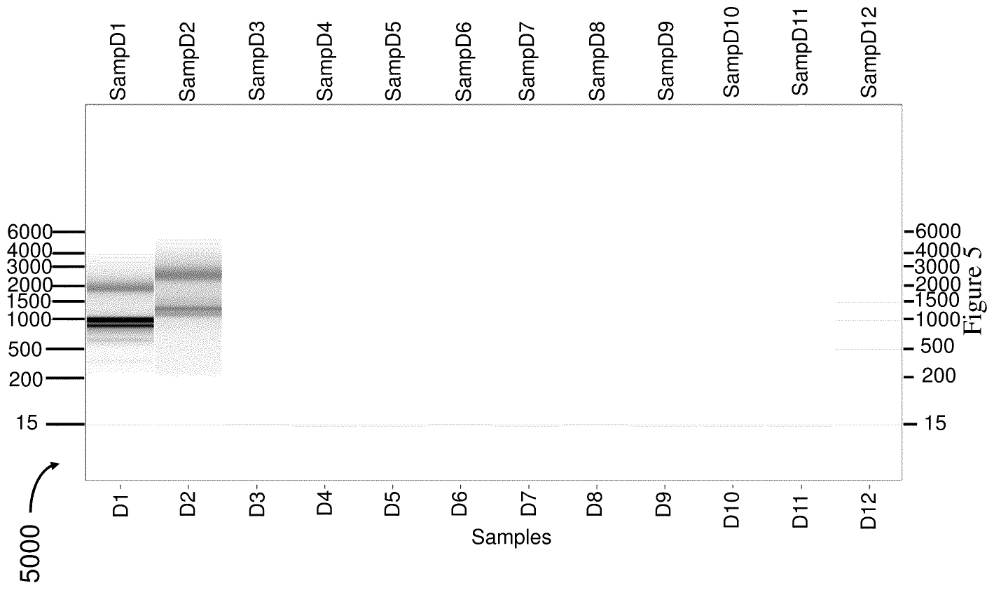

FIGS. 4A, 4B and 4C showed the Urea-PAGE results of the reacted RNA products after adding different nucleotide analogues to the 3′ termini of RNAs with C terminal base (Urea-PAGE result 4100 in FIG. 4A), G terminal base (Urea-PAGE result 4200 in FIG. 4B) and U terminal base (Urea-PAGE result 4300 in FIG. 4C) respectively. The results showed that poly(A) polymerase-based extension successfully added oligo 2-Amino-6-Cl-purine-rTP, 2-Amino-ATP and 2′-F-dATP to all the model short RNAs with different 3′ terminal bases, with 2-Amino-6-Cl-purine-rTP being the nucleotide analogue which produced the best results.

### Example 6: Addition of Oligo 2-Amino-6-Cl-Purine-rTP Sequence to the 3′ Termini of the Model RNA Mixture by Poly(A) Polymerase-Based Extension

### Materials and Methods

In this example, three model mRNAs CLuc-53AGGCCCCU, EGFP-40A, and UnaG-120A as shown in Table 1 are mixed in an equal amount to create a model mRNA mixture. The same poly(A) polymerase-based extension as described in Example 5 was used to add 2-Amino-6-Cl-purine-rTP to the model RNA mixture. Fragment Analyzer (Agilent®) was used to measure the lengths of the model RNA mixture before and after the reaction.

### Results

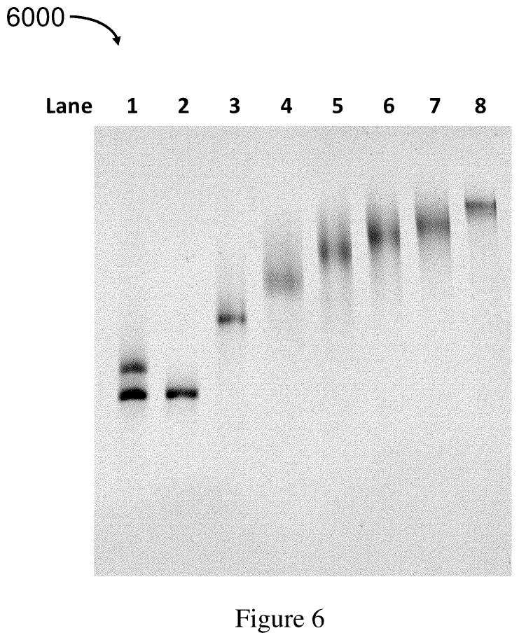

Referring now to graph 5000 in FIG. 5. SampD1 is the original model mRNA mixture; SampD2 is the model mRNA mixture with 15-minute 2-Amino-6-Cl-purine-rTP extension; SampD12 is the RNA ladder. As shown in FIG. 5, the upward shift of all three bands in the after-reaction sample (SampD2) compared to the original model RNA mixture (SampD1) indicated that oligo 2-Amino-6-Cl-purine-rTP sequence was added to all model mRNAs in the model RNA mixture.

Together with Example 5, these results showed the addition of oligo 2-Amino-6-Cl-purine-rTP sequence to the 3′ terminus of RNA is unbiased to the 3′ terminal base of the RNA.

### Example 7: Sequential Addition of Oligo 2-Amino-6-Cl-Purine-rTP Sequence and Oligo(A) Sequence to the 3′ Terminal Base of the RNA by One-Pot 3′ Extension

### Materials and Methods

In this example, the reaction cocktail for the one-pot 3′ extension of model short RNA (as shown in Table 1) was prepared as described in Table 4. The reaction cocktail (without ATP) was incubated at 37° C. for 15 minutes to allow for 2-Amino-6-Cl-purine-rTP addition. After 15 minutes of reaction, 1 μL of 10 mM ATP (New England BioLabs®) was directly added into the reaction mixture, and further incubated for different durations of time at 37° C.

TABLE 4
One-pot 3′ extension reaction cocktail

| | | Final | | Time of |
|---|---|---|---|---|
| Component | Volume (μL) | concentration/amount | | addition |
| RNA sample | | 100 ng of total RNA | | Beginning of |
| | | | | reaction |
| Poly(A) polymerase reaction | 1 | 1x | | Beginning of |
| buffer (10x) (B0276SVIAL, | | | | reaction |
| New England BioLabs ®) | | | | |
| 2-Amino-6-Cl-purine-rTP (10 | 1 | 1 | mM | Beginning of |
| mM) (Trilink Biotechnologies) | | | | reaction |
| Water | Top up to the | | | Beginning of |
| | final volume | | | reaction |
| | of 10 μL | | | |
| E coli. poly(A) polymerase | 0.5 | 2.5 | units | Beginning of |
| (5000 units/mL, M0276SVIAL, | | | | reaction |
| New England BioLabs ®) | | | | |
| ATP | 1 | 0.9 | mM | 15 minutes |

after reaction

### Results

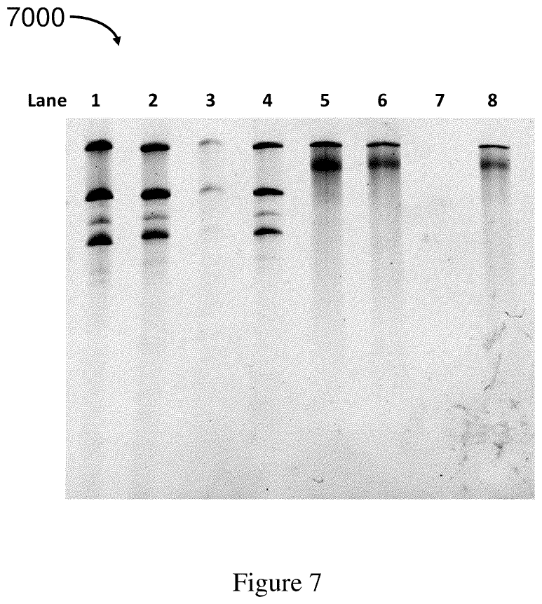

FIG. 6 shows the Urea-PAGE result 6000 illustrating the time-dependent oligo An extension to the first tailed RNA carrying oligo 2-Amino-6-Cl-purine-rTP sequence. Lane 1 shows the 136 nt, 156 nt RNA ladder; Lane 2 shows the original model short RNA; Lane 3 shows the first tailed model short RNA with 15-minute 2-Amino-6-Cl-purine-rTP addition; Lane 4 shows the second tailed model short RNA with 15-minute 2-Amino-6-Cl-purine-rTP addition followed by 5-minute ATP addition; Lane 5 shows the second tailed model short RNA with 15-minute 2-Amino-6-Cl-purine-rTP addition followed by 10-minute ATP addition; Lane 6 shows the second tailed model short RNA with 15-minute 2-Amino-6-Cl-purine-rTP addition followed by 15-minute ATP addition; Lane 7 shows the second tailed model short RNA with 15-minute 2-Amino-6-Cl-purine-rTP addition followed by 20-minute ATP addition; Lane 8 shows the model short RNA with 20-minute ATP addition.

The results showed that one-pot 3′ extension successfully added oligo 2-Amino-6-Cl-purine-rTP sequence and oligo(A) sequence to the model short RNA sequentially.

### Example 8: Unbiased Enrichment of mRNAs in dT Adapter Ligation by One-Pot 3′ Extension

### Materials and Methods

Table 5 shows the single-stranded oligos to construct the sequencing adapter (also referred to as “dT adapter”) synthesized by Integrated DNA Technologies IDT® and Ruibo Biotech (Guangzhou) respectively. The dT adapter was internally biotinylated. To pre-anneal the oligos to construct the dT adapter, 10 mM of the two oligos were pre-annealed in the annealing buffer (10 μM Tris-HCl, 50 mM NaCl, 0.1 mM EDTA) by incubating at 95° C. for 2 minutes and −0.1° C./s to 25° C. 100 ng of RNA sample was ligated following Table 6 at 20° C. for 1 hour. The successfully ligated RNA molecules were bound using Dynabeads™ Myone™ Streptavidin C1 magnetic beads (Invitrogen™) following the manufacturer's protocol. The bound RNA was disassociated in nuclease-free water by heating at a rate of 0.5° C./s to 70° C. and cooling to room temperature. The bound or unbound RNA solution was purified using the Monarch RNA cleanup kit (New England BioLabs®).

The model mRNA mixture (mixture of CLuc-53AGGCCCCU, EGFP-40A, and UnaG-120A as shown in Table 1) was directly treated with dT adapter ligation or undergo the one-pot 3′ extension following the reaction cocktail in Table 4 before dT adapter ligation. The RNA products after ligation were treated with Dynabeads™ Myone™ Streptavidin C1 magnetic beads as described above. The reacted RNA products were visualized using Urea-PAGE.

TABLE 5
Oligo nucleotides used to construct
the dT adapter

| Oligo | |
|---|---|
| nucleotides | Sequence |
| Adapter | P¹-CTGTCTCTTATACACATCTGACGCT(bio)²GC |
| oligo | CGACGA (SEQ ID NO: 8) |
| Oligo(dT) | TCGTCGGCAGCGTCAGATGTGTATAAGAGACAGTTT |
| oligo | TTTTTTT (SEQ ID NO: 9) |

¹the 5′ of the model adapter carries monophosphate.
²the bolded T was labeled with biotin.

TABLE 6
dT adapter ligation reaction cocktail

| | | Final | |
|---|---|---|---|
| | Volume | concentration/ | |
| Component | (μL) | amount | |
| RNA sample | | 100 ng of | |
| | | total RNA | |
| dT adapter (10 μM) | 2.29 | 1.143 | μM |
| T4 DNA Ligase Reaction | 2 | 1x | |
| Buffer (10x) (B0202SVIAL, | | | |
| New England BioLabs ®) | | | |
| Water | Top up to the | | |
| | final volume | | |
| | of 20 μL | | |
| T4 DNA Ligase (400,000 | 1 | 400 | units |

units/mL, M0202SVIAL,
New England BioLabs ®)

### Results

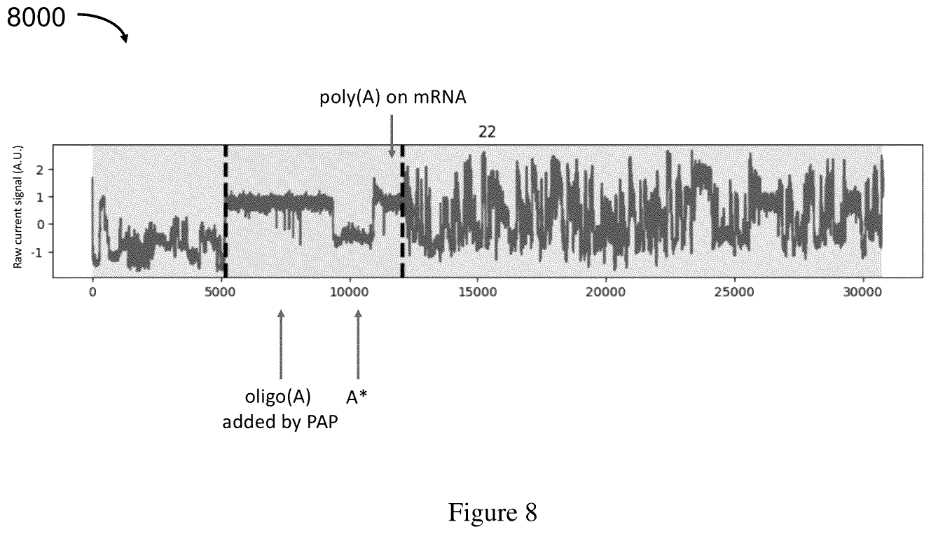

Referring now to FIG. 7, showing the Urea-PAGE result 7000 of mRNA samples treated with dT adapter ligation with or without prior one-pot 3′ extension reaction. Lane 1 shows the original model mRNA mixture; Lane 2 shows the reaction product of model mRNA mixture ligated with dT adapter; Lane 3 shows the RNA left-over in the solution after streptavidin beads-based RNA pull-down of Lane 2; Lane 4 shows RNA captured on streptavidin-beads in RNA pull-down of Lane 2; Lane 5 shows model mRNA mixture with 15-minute 2-Amino-6-Cl-purine-rTP addition followed by 15-minute ATP addition; Lane 6 shows reaction product of Lane 5 ligated with dT adapter; Lane 7 shows RNA left-over in the solution after streptavidin beads-based RNA pull-down (˜0%) of Lane 6; Lane 8 shows the RNA captured on streptavidin-beads in RNA pull-down (˜100%) of Lane 6.

FIG. 7 showed that the model mRNA mixture directly treated with dT adapter ligation cannot be completely captured by the beads (as shown in Lanes 3 and 4). In contrast, the model mRNA mixture that undergoes the one-pot 3′ extension before dT adapter ligation can be fully captured by the beads (as shown in Lanes 7 and 8). These results indicated the one-pot 3′ extension enables unbiased dT adapter ligation to enhance mRNA capture yield.

### Example 9: Detection of the Oligo Nucleotide Analogue Sequence and the Oligo(A) Sequence of One-Pot 3′ Extension Reaction Product by Nanopore Direct RNA Sequencing

### Materials and Methods

One-pot 3′ extension was performed on 500 ng of model RNA mixture (mixture of CLuc-53AGGCCCCU, EGFP-40A, and UnaG-120A as shown in Table 1) with a 5× scale-up of the reaction cocktail shown in Table 4. After the 15 minutes of 2-Amino-6-Cl-purine-rTP addition, 1 μL of 10 mM ATP (New England BioLabs®) was directly added into the reaction mixture and further incubated for 15 minutes at 37° C. The reaction mixture is purified before undergoing the Nanopore direct RNA sequencing following the standard protocol of The Direct RNA Sequencing Kit (SQK-RNA002). The model RNA mixture without treatment was sequenced as a negative control.

### Results

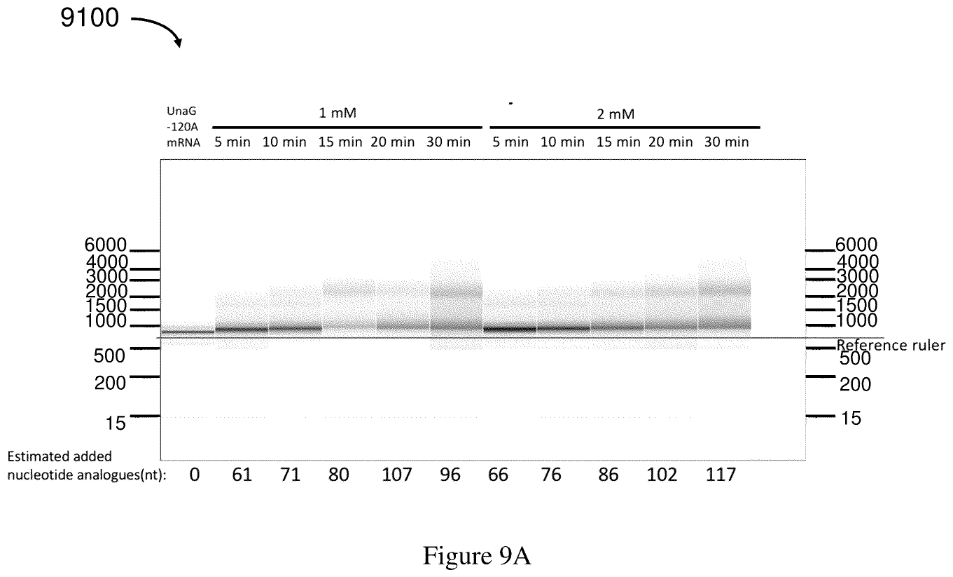

Referring now to FIG. 8, graph 8000 showed the raw current signal of direct RNA sequencing of the one pot 3′ extension reaction product. The result showed that the oligo nucleotide analog sequence of the reacted RNA product can be distinguished with a continuous lower signal between the poly(A) tail of the mRNA and the oligo(A) added by the poly(A) polymerase. This signal pattern confirmed that the oligo nucleotide analogue sequence and the oligo(A) sequence added to the RNA sample by one pot 3′ extension can be detected by Nanopore direct RNA sequencing. As such, the oligo nucleotide analogue sequence can serve as a marker to locate the last base of the 3′ tail of individual RNA molecule in sequencing, so the intact 3′ tail sequence of the individual RNA molecule can be correctly mapped.

Basecalling was performed on the RNA mixture with or without the one pot 3′ extension treatment by the default Guppy algorithm and mapping to the mRNA sequence. Results showed that the percentage of successfully mapped Cluc-53AGGCCCCU mRNA increased from 3.7% for the samples without any treatment to 10.5% for the sample with the one-pot 3′ extension treatment. This result indicated that the one-pot 3′ extension can enrich the mRNAs with non-A terminus for direct RNA sequencing.

### Example 10: Optimization of the Addition of Nucleotide Analogues by Increasing the Nucleotide Analogue Concentration and Incubation Time in Poly(A) Polymerase-Based Reaction

### Materials and Methods

In this example, the optimized conditions for the addition of nucleotide analogues to the three model mRNAs UnaG-120A, EGFP-40A and CLuc-53AGGCCCCU as shown in Table 1 by poly(A) polymerase-based reaction were explored. Table 7 shows the reaction cocktail for the optimization of the addition of nucleotide analogues by poly(A) polymerase-based reaction. The 10 μL of the reaction mixture was incubated at 37° C. for different incubation time, i.e. for 5, 10, 15, 20 or 30 minutes. The reacted RNA products were visualized using Fragment Analyzer.

TABLE 7
Poly(A) polymerase reaction cocktail for optimization
of the addition of nucleotide analogue

| | | Final |
|---|---|---|
| | Volume | concentration/ |
| Component | (μL) | amount |
| RNA sample | | 100 ng of total RNA |
| Poly(A) polymerase reaction | 1 | 1x |
| buffer (10x) (B0276SVIAL, | | |
| New England BioLabs ®) | | |
| 2-Amino-6-Cl-purine-rTP (10, | 1 | 1, 2, 4, or 8 mM |
| 20, 40, or 80 mM) (Trilink | | |
| Biotechnologies) | | |
| Water | Top up to the | |
| | final volume | |
| | of 10 μL | |
| E coli. poly(A) polymerase | 0.5 | 2.5 units |
| (5000 units/mL, M0276SVIAL, | | |
| New England BioLabs ®) | | |

### Results

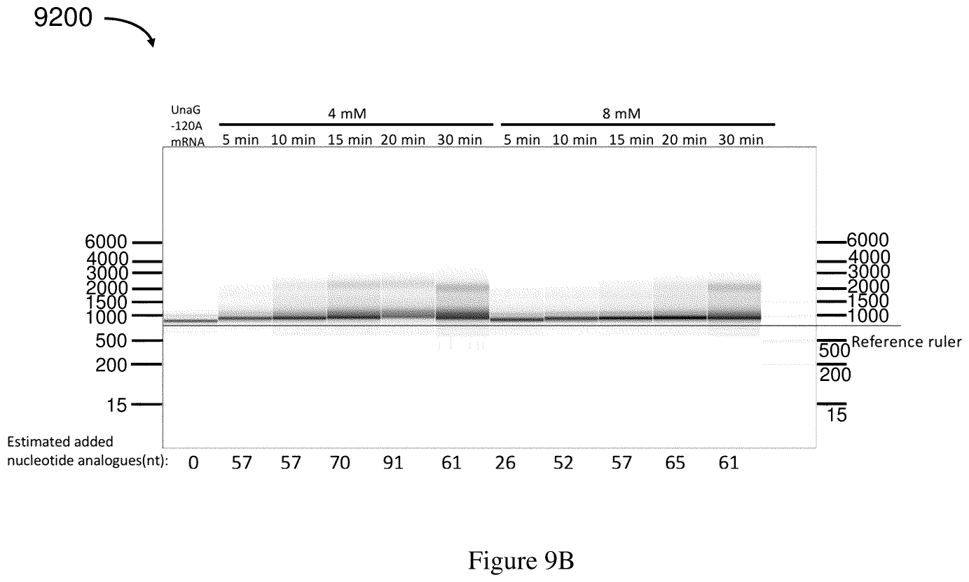

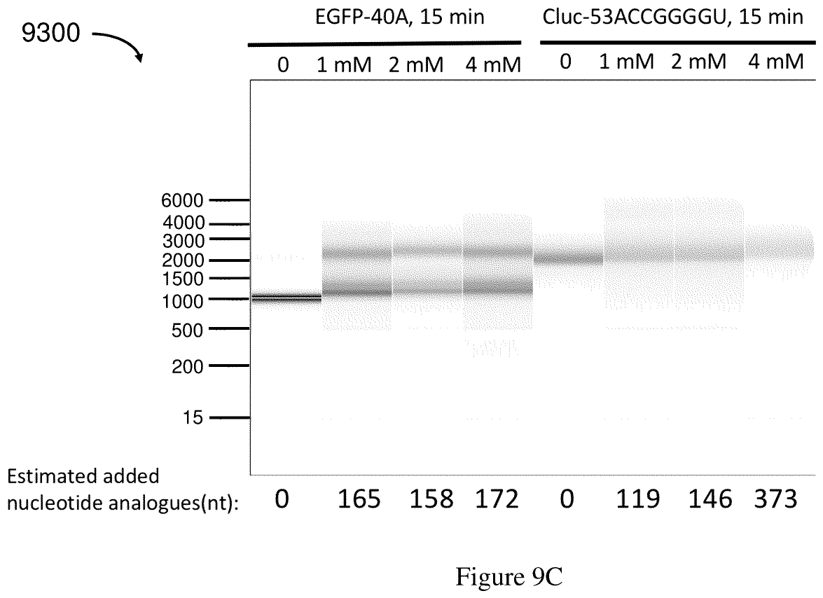

Referring now to FIGS. 9A and 9B, graphs 9100 and 9200 show reacted RNA products with different incubation time (5, 10, 15, 20 or 30 minutes) and different nucleotide analogue concentrations (1 mM and 2 mM in graph 9100, 4 mM and 8 mM in graph 9200) for adding nucleotide analogues to the 3′ termini of UnaG-120A mRNA. The results showed that on UnaG-120A RNA, increasing the nucleotide analogue concentration to 2 mM and 4 mM results in a more efficient addition of the oligo nucleotide analogue sequence, while 8 mM showed a lower efficiency in the reaction. When it is less than 20 minutes, there is a positive correlation between the additional length of oligo nucleotide analogue and time. For some conditions, no elongation was observed for longer incubation time than 20 minutes, suggesting the reaction reached the equilibrium.

Referring now to FIG. 9C, graph 9300 shows the increase of nucleotide analogue concentration (from 1 mM to 2 mM and 4 mM) results in an increased addition efficiency onto the other two model mRNAs EGFP-40A and CLuc-53AGGCCCCU. These results indicate that the efficiency of oligo nucleotide analogue addition can be improved by increasing the nucleotide analogue and reaction time. Notably, no significant RNA degradation was observed in the reaction products, indicating the reaction has little-to-no effect to the integrity of RNA samples.

### Example 11: Addition of Different Nucleotide Analogues to the 3′ Termini of RNA as the Oligo Nucleotide Analogue Sequence by Poly(A) Polymerase-Based Reaction

### Materials and Methods

In this example, the ability of poly(A) polymerase-based reaction to add different nucleotide analogues to the 3′ termini of UnaG-120A mRNA as the oligo nucleotide analogue sequence has been tested. Table 8 shows the reaction cocktail for testing two different nucleotide analogues, 2-Amino-ATP and 2-Amino-6-Cl-purine-rTP, as well as their combinations in the poly(A) polymerase-based reaction. The 10 μL of the reaction mixture was incubated at 37° C. for 15 minutes. The reacted RNA products were visualized using Fragment Analyzer.

TABLE 8
Poly(A) polymerase-based reaction cocktail for testing different
nucleotide analogues and their combination thereof

| | | Final | |
|---|---|---|---|
| | Volume | concentration/ | |
| Component | (μL) | amount | |
| UnaG-120A RNA | | 100 | ng |
| Poly(A) polymerase reaction | 1 | 1x | |
| buffer (10x) (B0276SVIAL, | | | |
| New England BioLabs ®) | | | |
| 2-Amino-ATP (10 mM), 2- | 1 | 1 mM nucleotide | |
| Amino-6-Cl-purine-rTP | | analogue | |
| (10mM), or mixture of 2- | | (NTP form) | |
| Amino-ATP (5 mM) and 2- | | | |
| Amino-6-Cl-purine-rTP (5 | | | |
| mM) (Trilink Biotechnologies) | | | |
| Water | Top up to the final | | |
| | volume of 10 μL | | |
| E coli. poly(A) polymerase | 0.5 | 2.5 | units |

(5000 units/mL, M0276SVIAL,
New England BioLabs ®)

### Results

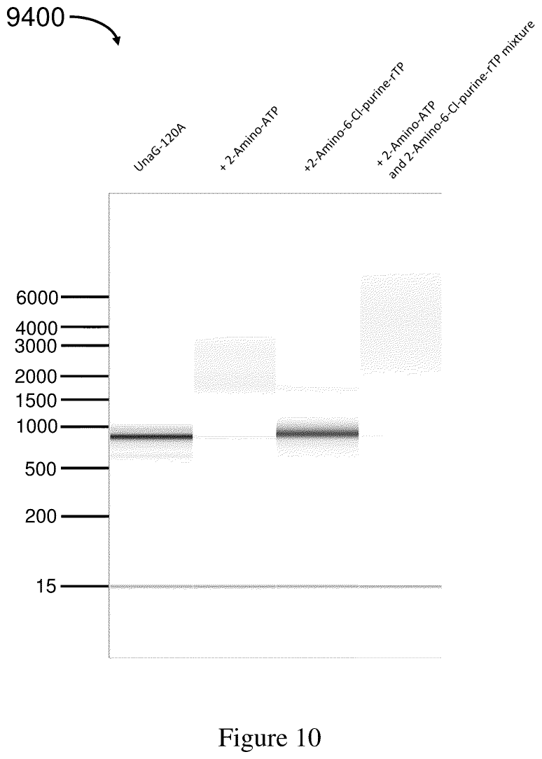

Referring now to FIG. 10, graph 9400 shows that both 2-Amino-ATP, 2-Amino-6-Cl-purine-rTP, and the mixture of 2-Amino-ATP and 2-Amino-6-Cl-purine-rTP can be added to the UnaG-120A mRNA by poly(A) polymerase. The product of adding 2-Amino-ATP has a distribution of RNA sizes shown as a smear, while the product of adding 2-Amino-6-Cl-purine-rTP has an upshifted clear main band compared with the non-treated sample. Notably, the product of adding the mixture of 2-Amino-ATP and 2-Amino-6-Cl-purine-rTP shows a distribution of RNA sizes similar to but longer than the 2-Amino-ATP-added RNA product, confirming the added sequence was a combination of the two nucleotide analogues.

### Example 12: Examples of Nucleotide Analogue that can be Used in the One-Pot 3′ Extension Reaction

Table 9 lists the examples of nucleotide analogue that can be used in the one-pot 3′ extension reaction. It will be appreciated that the list of example nucleotide analogues in Table 9 is non-exhaustive as other nucleotide analogues can also be used in the one-pot 3′ extension and would fall within the scope of the present invention.

TABLE 9
Examples of nucleotide analogue that can be used in the one-pot 3′ extension reaction

| Name | Structure (in nucleoside triphosphates form) |
|---|---|
| 2-Amino-ATP | 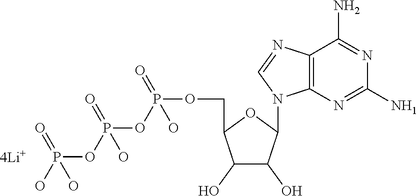 [SMILES: Nc1nc(N)c2ncn(C3OC(COP(=O)(O)OP(=O)(O)OP(=O)(O)O)C(O)C3O)c2n1] |
| 2-Amino-6-Cl- purine-rTP | 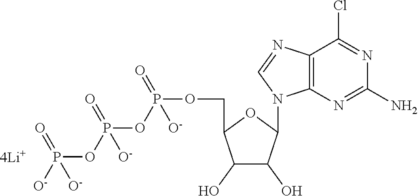 [SMILES: Nc1nc(Cl)c2ncn(C3OC(COP(=O)([O-])OP(=O)([O-])OP(=O)([O-])[O-])C(O)C3O)c2n1] |
| 2′-F-dATP | 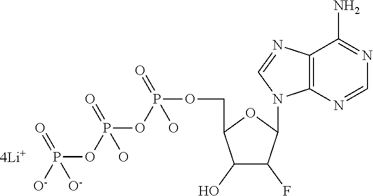 [SMILES: Nc1ncnc2c1ncn2C1OC(COP(=O)(O)OP(=O)(O)OP(=O)([O-])[O-])C(O)C1F] |
| 2′-Azido-dATP | 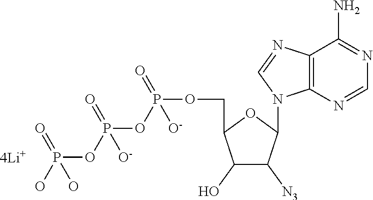 [SMILES: [N-]=[N+]=NC1C(O)C(COP(=O)([O-])OP(=O)([O-])OP(=O)(O)O)OC1n1cnc2c(N)ncnc21] |
| 7-Deaza-ATP | 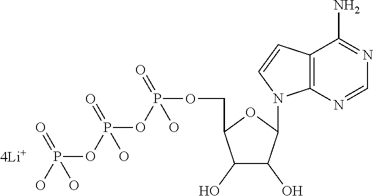 [SMILES: Nc1ncnc2c1ccn2C1OC(COP(=O)(O)OP(=O)(O)OP(=O)(O)O)C(O)C1O] |
| 2-Cl-ATP | 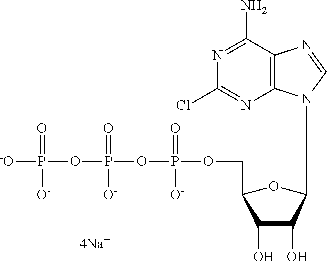 |
| 2′-Bromo-dATP | 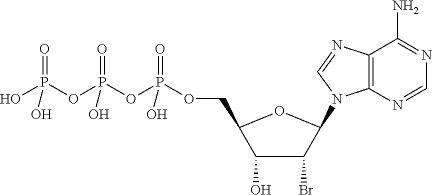 [SMILES: Nc1ncnc2c1ncn2[C@@H]1O[C@H](COP(=O)(O)OP(=O)(O)OP(=O)(O)O)[C@@H](O)[C@H]1Br] |
| 2-Fluoro-ATP | 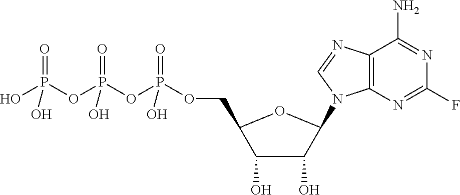 [SMILES: Nc1nc(F)nc2c1ncn2[C@@H]1O[C@H](COP(=O)(O)OP(=O)(O)OP(=O)(O)O)[C@@H](O)[C@H]1O] |
| 8-Bromo-ATP | 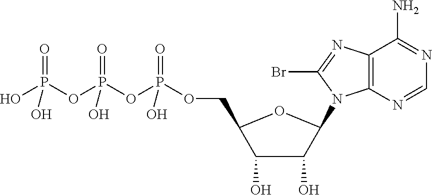 [SMILES: Nc1ncnc2c1nc(Br)n2[C@@H]1O[C@H](COP(=O)(O)OP(=O)(O)OP(=O)(O)O)[C@@H](O)[C@H]1O] |
| 8-Iodo-ATP | 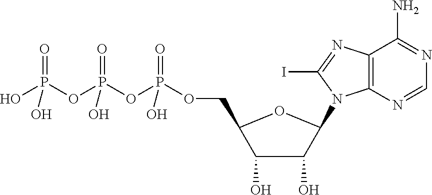 [SMILES: Nc1ncnc2c1nc(I)n2[C@@H]1O[C@H](COP(=O)(O)OP(=O)(O)OP(=O)(O)O)[C@@H](O)[C@H]1O] |
| 2′-Iodo-dATP | 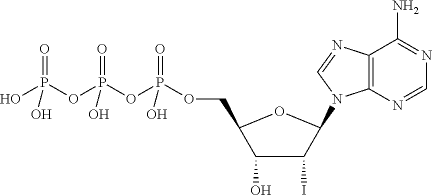 [SMILES: Nc1ncnc2c1ncn2[C@@H]1O[C@H](COP(=O)(O)OP(=O)(O)OP(=O)(O)O)[C@@H](O)[C@H]1I] |
| 1-Thio-UTP | 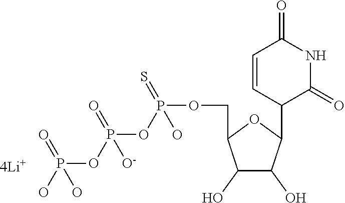 [SMILES: O=C1C=CC(C2OC(COP(O)(=S)OP(=O)([O-])OP(=O)(O)O)C(O)C2O)C(=O)N1] |
| 1-Thio-GTP | 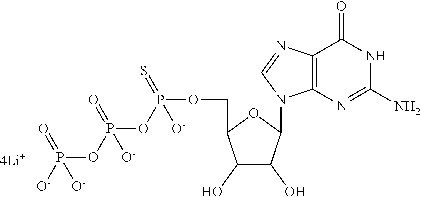 [SMILES: Nc1nc2c(ncn2C2OC(COP([O-])(=S)OP(=O)([O-])OP(=O)([O-])[O-])C(O)C2O)c(=O)[nH]1] |
| 1-Thio-CTP | 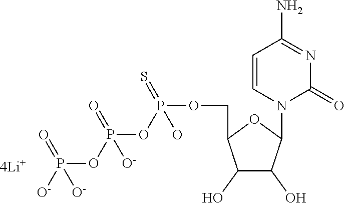 [SMILES: Nc1ccn(C2OC(COP(O)(=S)OP(=O)([O-])OP(=O)([O-])[O-])C(O)C2O)c(=O)n1] |
| 1-Thio-ATP | 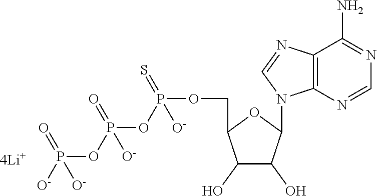 [SMILES: Nc1ncnc2c1ncn2C1OC(COP([O-])(=S)OP(=O)([O-])OP(=O)([O-])[O-])C(O)C1O] |
| 1-Thio-dTTP | 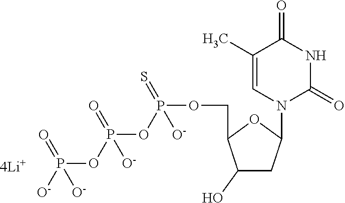 [SMILES: Cc1cn(C2CC(O)C(COP([O-])(=S)OP(=O)([O-])OP(=O)([O-])[O-])O2)c(=O)[nH]c1=O] |
| 1-Thio-dGTP | 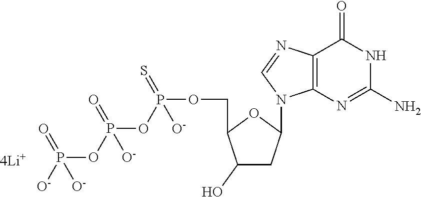 [SMILES: Nc1nc2c(ncn2C2CC(O)C(COP([O-])(=S)OP(=O)([O-])OP(=O)([O-])[O-])O2)c(=O)[nH]1] |
| 1-Thio-dCTP | 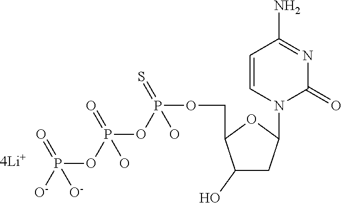 [SMILES: Nc1ccn(C2CC(O)C(COP(O)(=S)OP(=O)(O)OP(=O)([O-])[O-])O2)c(=O)n1] |
| 1-Thio-dATP | 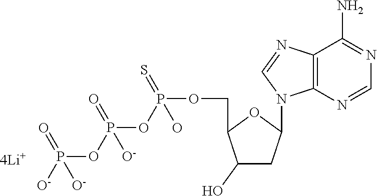 [SMILES: Nc1ncnc2c1ncn2C1CC(O)C(COP(O)(=S)OP(=O)([O-])OP(=O)([O-])[O-])O1] |
| 5-(Cyanine 7)-UTP | 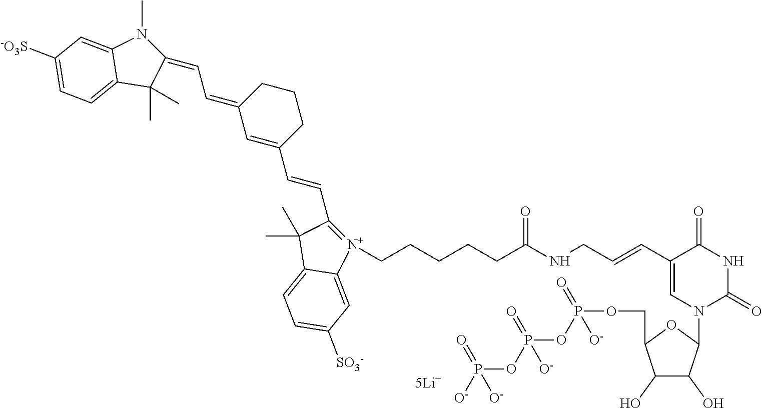 |
| Cyanine 5-UTP | 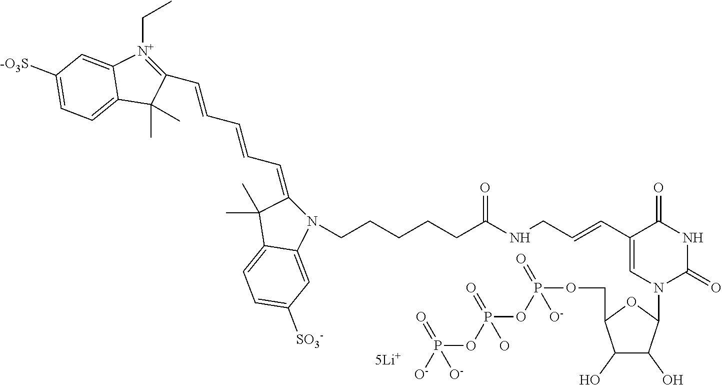 |
| 6-(Cyanine 5)-CTP | 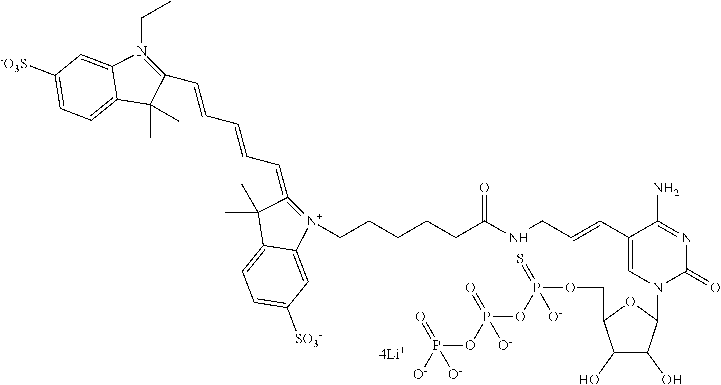 |
| 6-(Cyanine 3)-UTP | 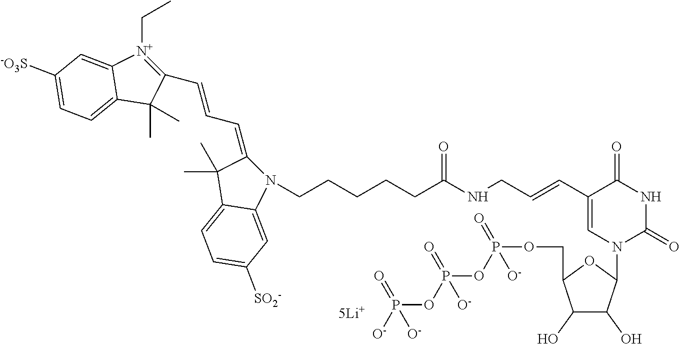 |
| 6-(Cyanine 3)-CTP | 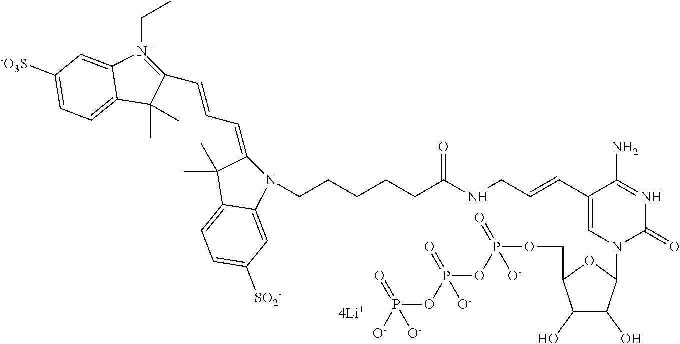 |
| Cyanine 5-dUTP | 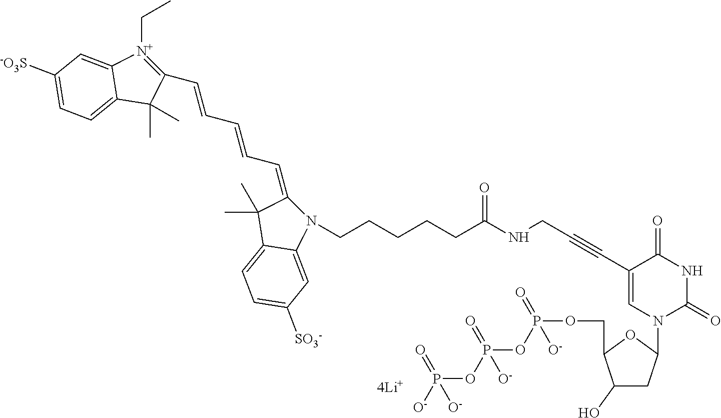 |
| Cyanine 5-dCTP | 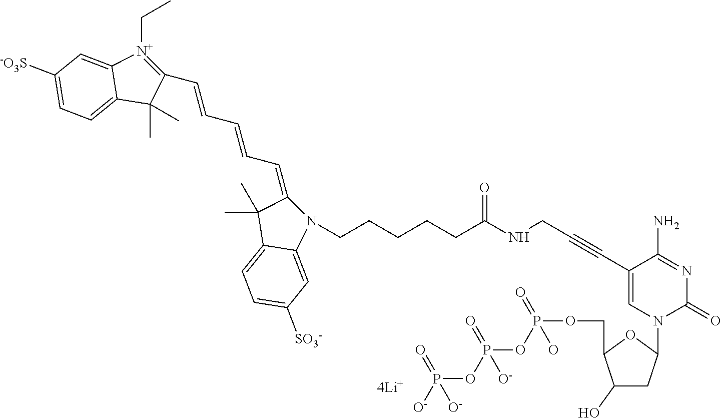 |
| Cyanine 3-dUTP | 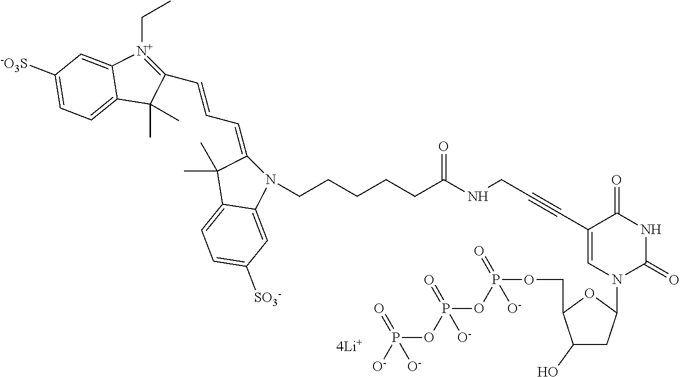 |
| Cyanine 3-dCTP | 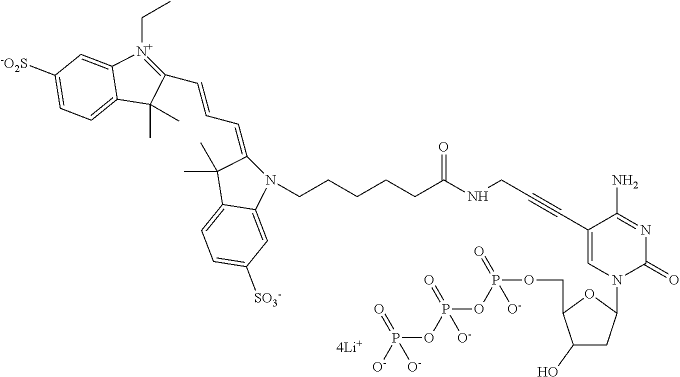 |
| Biotin-16-7- Deaza-dGTP | 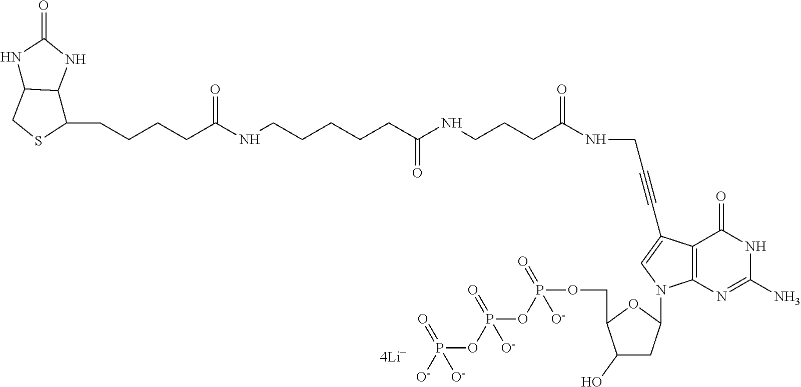 [SMILES: Nc1nc2c(c(C#CCNC(=O)CCCNC(=O)CCCCCNC(=O)CCCCC3SCC4NC(=O)NC43)cn2C2CC(O)C(COP(=O)([O-])OP(=O)([O-])OP(=O)([O-])[O-])O2)c(=O)[nH]1] |
| Desthiobiotin- 16-UTP | 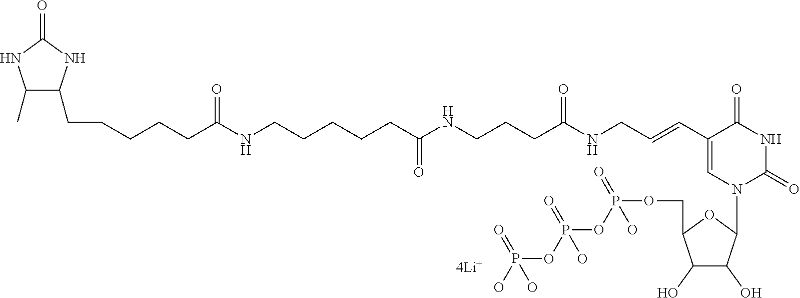 [SMILES: CC1NC(=O)NC1CCCCCC(=O)NCCCCCC(=O)NCCCC(=O)NC/C=C/c1cn(C2OC(COP(=O)(O)OP(=O)(O)OP(=O)(O)O)C(O)C2O)c(=O)[nH]c1=O] |
| Desthiobiotin- 6-dCTP | 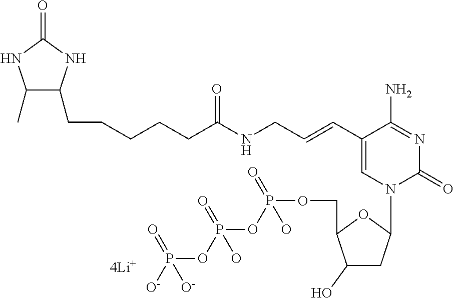 [SMILES: CC1NC(=O)NC1CCCCCC(=O)NC/C=C/c1cn(C2CC(O)C(COP(=O)(O)OP(=O)(O)OP(=O)([O-])[O-])O2)c(=O)nc1N] |
| Dabcyl-5-3- Aminoallyl- 2′-dUTP | 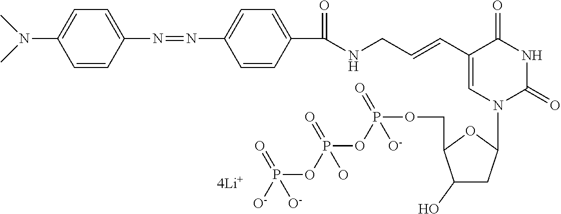 [SMILES: CN(C)c1ccc(N=Nc2ccc(C(=O)NC/C=C/c3cn(C4CC(O)C(COP(=O)([O-])OP(=O)(O)OP(=O)([O-])[O-])O4)c(=O)[nH]c3=O)cc2)cc1] |
| Biotin-16- AA-UTP | 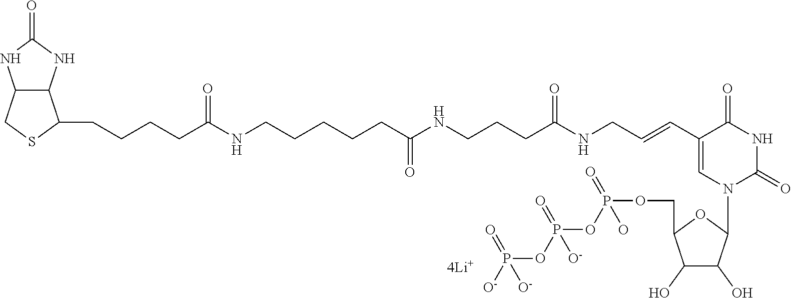 [SMILES: O=C(CCCNC(=O)CCCCCNC(=O)CCCCC1SCC2NC(=O)NC21)NC/C=C/c1cn(C2OC(COP(=O)(O)OP(=O)([O-])OP(=O)([O-])[O-])C(O)C2O)c(=O)[nH]c1=O] |
| N4-Biotin- dCTP | 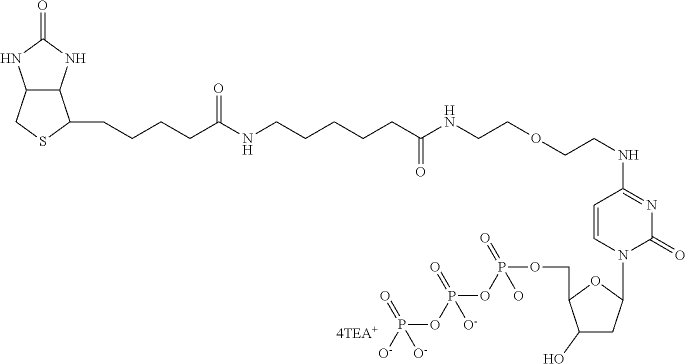 [SMILES: O=C(CCCCC1SCC2NC(=O)NC21)NCCCCCC(=O)NCCOCCNc1ccn(C2CC(O)C(COP(=O)(O)OP(=O)([O-])OP(=O)([O-])[O-])O2)c(=O)n1] |
| Biotin-16- AA-CTP | 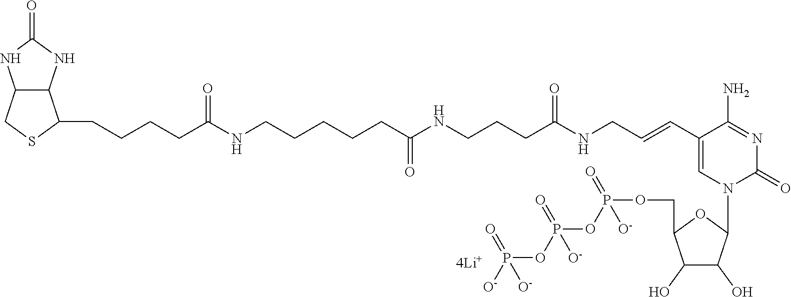 [SMILES: Nc1nc(=O)n(C2OC(COP(=O)([O-])OP(=O)([O-])OP(=O)([O-])[O-])C(O)C2O)cc1/C=C/CNC(=O)CCCNC(=O)CCCCCNC(=O)CCCCC1SCC2NC(=O)NC21] |
| Biotin-16- AA-dCTP | 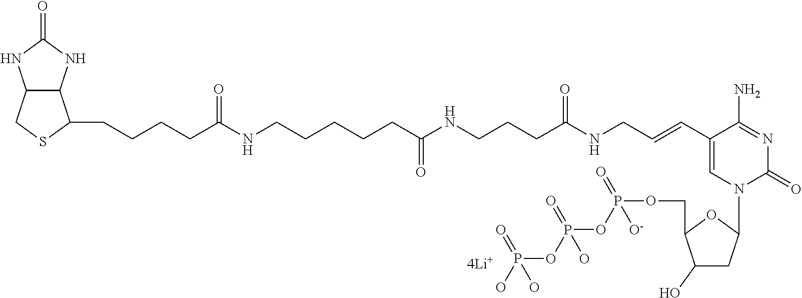 [SMILES: Nc1nc(=O)n(C2CC(O)C(COP(=O)([O-])OP(=O)(O)OP(=O)(O)O)O2)cc1/C=C/CNC(=O)CCCNC(=O)CCCCCNC(=O)CCCCC1SCC2NC(=O)NC21] |
| Biotin-16- AA-dUTP | 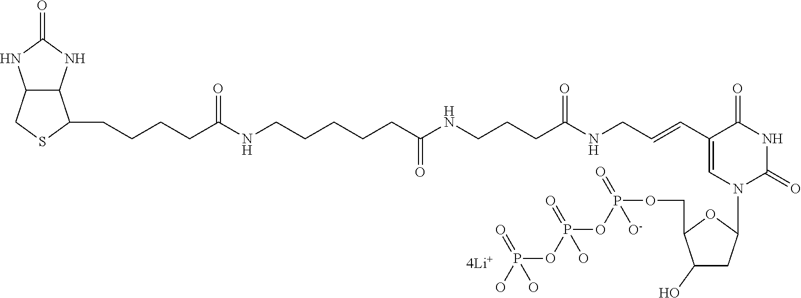 [SMILES: O=C(CCCNC(=O)CCCCCNC(=O)CCCCC1SCC2NC(=O)NC21)NC/C=C/c1cn(C2CC(O)C(COP(=O)([O-])OP(=O)(O)OP(=O)(O)O)O2)c(=O)[nH]c1=O] |
| dTTP | 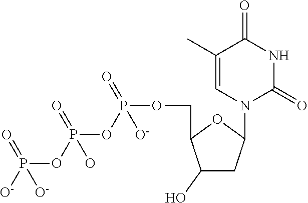 [SMILES: Cc1cn(C2CC(O)C(COP(=O)([O-])OP(=O)(O)OP(=O)([O-])[O-])O2)c(=O)[nH]c1=O] |
| dGTP | 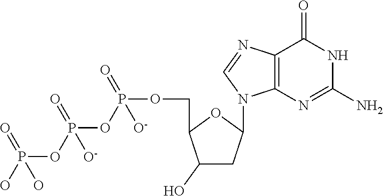 [SMILES: Nc1nc2c(ncn2C2CC(O)C(COP(=O)([O-])OP(=O)([O-])OP(=O)(O)O)O2)c(=O)[nH]1] |
| dCTP | 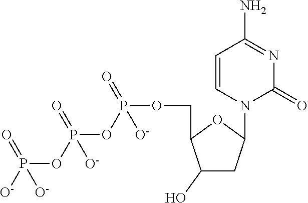 [SMILES: Nc1ccn(C2CC(O)C(COP(=O)([O-])OP(=O)([O-])OP(=O)([O-])[O-])O2)c(=O)n1] |
| dATP | 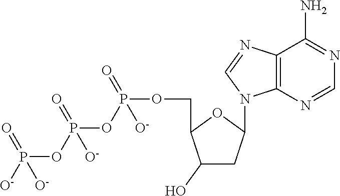 [SMILES: Nc1ncnc2c1ncn2C1CC(O)C(COP(=O)([O-])OP(=O)([O-])OP(=O)([O-])[O-])O1] |
| 7-Deaza-7- Propargyl- amino-dGTP | 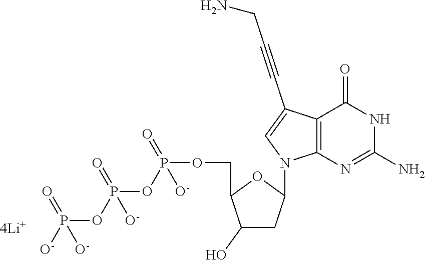 [SMILES: NCC#Cc1cn(C2CC(O)C(COP(=O)([O-])OP(=O)([O-])OP(=O)([O-])[O-])O2)c2nc(N)[nH]c(=O)c12] |
| 7-Deaza-7- Propargyl- amino-dATP | 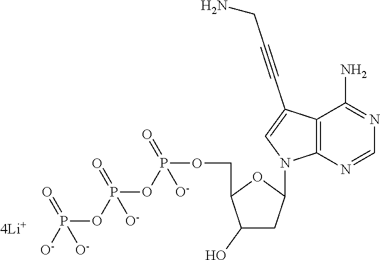 [SMILES: NCC#Cc1cn(C2CC(O)C(COP(=O)([O-])OP(=O)([O-])OP(=O)([O-])[O-])O2)c2ncnc(N)c12] |
| 5-Formyl- dUTP | 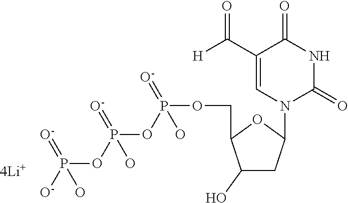 |
| 5-cadUTP | 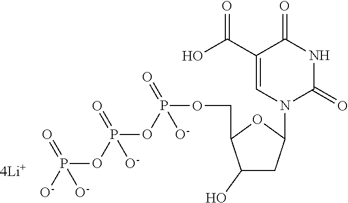 [SMILES: O=C(O)c1cn(C2CC(O)C(COP(=O)([O-])OP(=O)([O-])OP(=O)([O-])[O-])O2)c(=O)[nH]c1=O] |
| 5-Indolyl- AA-dUTP | 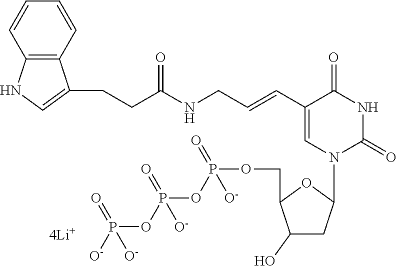 [SMILES: O=C(CCc1c[nH]c2ccccc12)NC/C=C/c1cn(C2CC(O)C(COP(=O)([O-])OP(=O)([O-])OP(=O)([O-])[O-])O2)c(=O)[nH]c1=O] |
| 5-Formyl- dCTP | 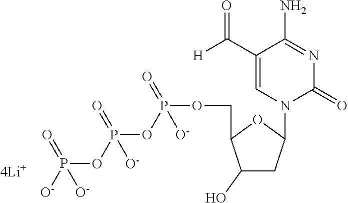 [SMILES: Nc1nc(=O)n(C2CC(O)C(COP(=O)([O-])OP(=O)([O-])OP(=O)([O-])[O-])O2)cc1C=O] |
| 5-Propargyl- amino-dUTP | 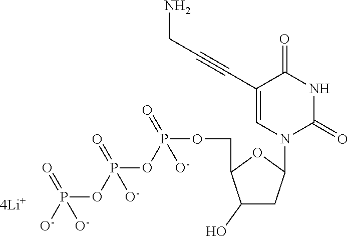 [SMILES: NCC#Cc1cn(C2CC(O)C(COP(=O)([O-])OP(=O)([O-])OP(=O)([O-])[O-])O2)c(=O)[nH]c1=O] |
| 5-Propargyl- amino-dCTP | 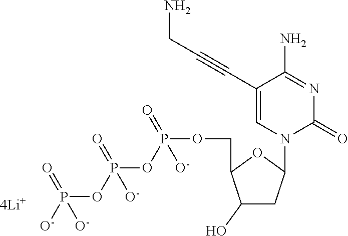 [SMILES: NCC#Cc1cn(C2CC(O)C(COP(=O)([O-])OP(=O)([O-])OP(=O)([O-])[O-])O2)c(=O)nc1N] |
| 5-Hydroxy- methyl-dUTP | 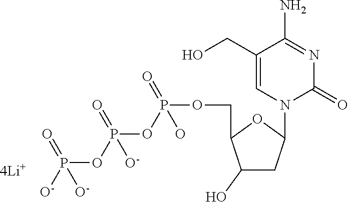 [SMILES: Nc1nc(=O)n(C2CC(O)C(COP(=O)(O)OP(=O)([O-])OP(=O)([O-])[O-])O2)cc1CO] |
| 2′-Deoxy- zebularine-TP | 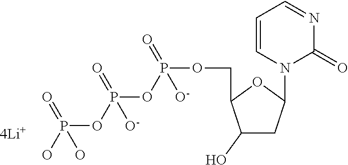 [SMILES: O=c1ncccn1C1CC(O)C(COP(=O)([O-])OP(=O)([O-])OP(=O)(O)O)O1] |
| N⁴-Methyl- dCTP | 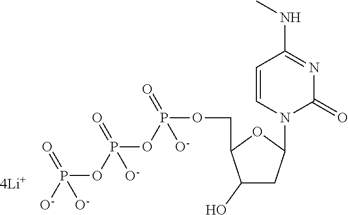 [SMILES: CNc1ccn(C2CC(O)C(COP(=O)([O-])OP(=O)([O-])OP(=O)([O-])[O-])O2)c(=O)n1] |
| 5-AA-dUTP | 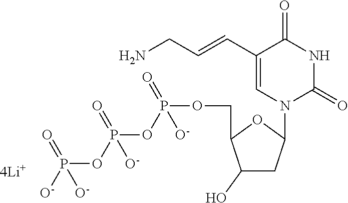 [SMILES: NC/C=C/c1cn(C2CC(O)C(COP(=O)([O-])OP(=O)([O-])OP(=O)([O-])[O-])O2)c(=O)[nH]c1=O] |
| 5-AA-dCTP | 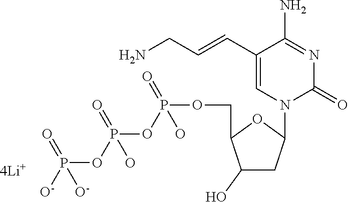 [SMILES: NC/C=C/c1cn(C2CC(O)C(COP(=O)(O)OP(=O)(O)OP(=O)([O-])[O-])O2)c(=O)nc1N] |
| 8-Chloro- dATP | 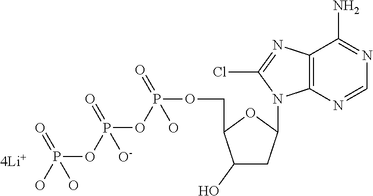 [SMILES: Nc1ncnc2c1nc(Cl)n2C1CC(O)C(COP(=O)(O)OP(=O)([O-])OP(=O)(O)O)O1] |
| 6-Thio-dGTP | 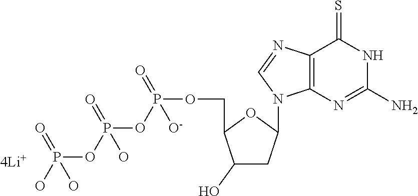 [SMILES: Nc1nc2c(ncn2C2CC(O)C(COP(=O)([O-])OP(=O)(O)OP(=O)(O)O)O2)c(=S)[nH]1] |
| 6-Aza-dUTP | 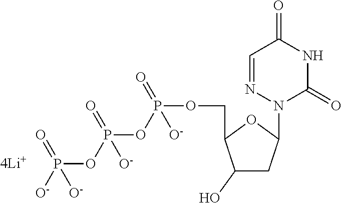 [SMILES: O=c1cnn(C2CC(O)C(COP(=O)([O-])OP(=O)([O-])OP(=O)([O-])[O-])O2)c(=O)[nH]1] |
| 2-Thio-dCTP | 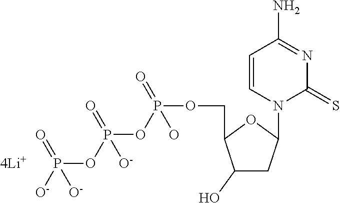 [SMILES: Nc1ccn(C2CC(O)C(COP(=O)(O)OP(=O)([O-])OP(=O)([O-])[O-])O2)c(=S)n1] |
| 4-Thio-dTTP | 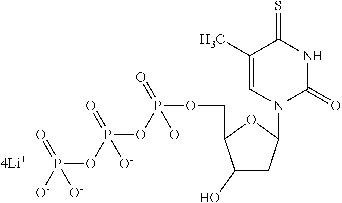 [SMILES: Cc1cn(C2CC(O)C(COP(=O)(O)OP(=O)([O-])OP(=O)([O-])[O-])O2)c(=O)[nH]c1=S] |
| 5-Hydroxy- dCTP | 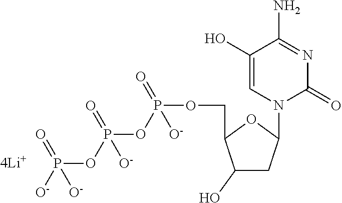 [SMILES: Nc1nc(=O)n(C2CC(O)C(COP(=O)([O-])OP(=O)([O-])OP(=O)([O-])[O-])O2)cc1O] |
| dPTP | 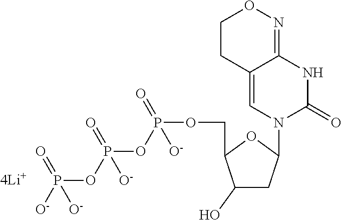 [SMILES: O=C1NC2=NOCCC2=CN1C1CC(O)C(COP(=O)([O-])OP(=O)([O-])OP(=O)([O-])[O-])O1] |
| 2-Thio-dTTP | 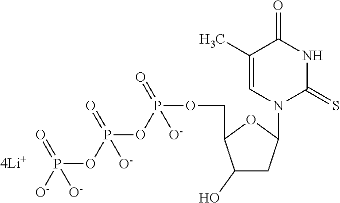 [SMILES: Cc1cn(C2CC(O)C(COP(=O)([O-])OP(=O)([O-])OP(=O)([O-])[O-])O2)c(=S)[nH]c1=O] |
| 8-Oxo-dGTP | 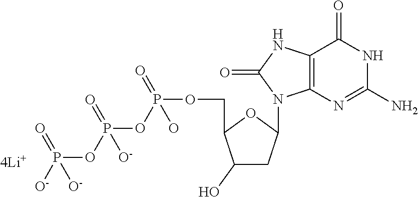 [SMILES: Nc1nc2c([nH]c(=O)n2C2CC(O)C(COP(=O)(O)OP(=O)([O-])OP(=O)([O-])[O-])O2)c(=O)[nH]1] |
| 8-Oxo-dATP | 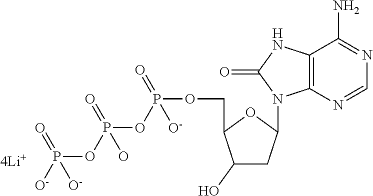 [SMILES: Nc1ncnc2c1[nH]c(=O)n2C1CC(O)C(COP(=O)([O-])OP(=O)(O)OP(=O)([O-])[O-])O1] |
| 5-Nitro-1- Indolyl-drTP | 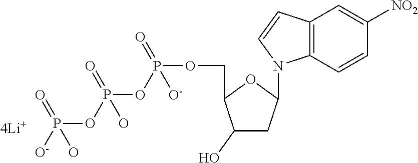 [SMILES: O=[N+]([O-])c1ccc2c(ccn2C2CC(O)C(COP(=O)([O-])OP(=O)(O)OP(=O)([O-])O)O2)c1] |
| N²-Methyl- dGTP | 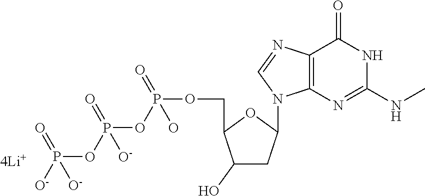 [SMILES: CNc1nc2c(ncn2C2CC(O)C(COP(=O)(O)OP(=O)([O-])OP(=O)([O-])[O-])O2)c(=O)[nH]1] |
| O⁶-Methyl- dGTP | 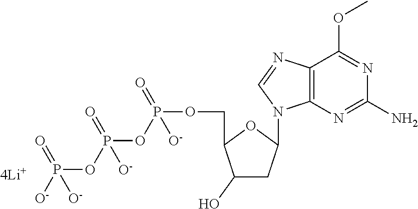 [SMILES: COc1nc(N)nc2c1ncn2C1CC(O)C(COP(=O)([O-])OP(=O)([O-])OP(=O)([O-])[O-])O1] |
| 5-Methyl- dCTP | 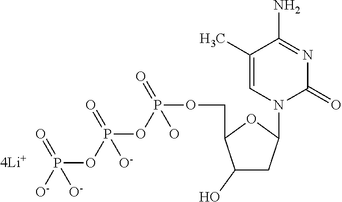 [SMILES: Cc1cn(C2CC(O)C(COP(=O)(O)OP(=O)([O-])OP(=O)([O-])[O-])O2)c(=O)nc1N] |
| N⁶-Methyl- dATP | 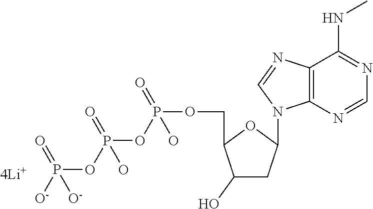 [SMILES: CNc1ncnc2c1ncn2C1CC(O)C(COP(=O)(O)OP(=O)(O)OP(=O)([O-])[O-])O1] |
| 5-Iodo-dUTP | 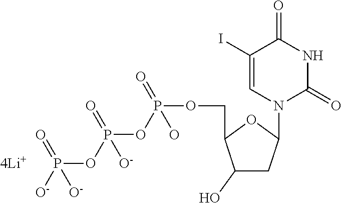 [SMILES: O=c1[nH]c(=O)n(C2CC(O)C(COP(=O)(O)OP(=O)([O-])OP(=O)([O-])[O-])O2)cc1I] |
| 5-Iodo-dCTP | 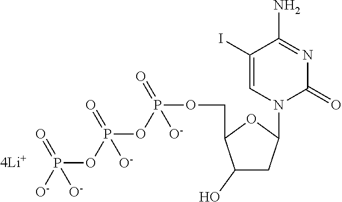 [SMILES: Nc1nc(=O)n(C2CC(O)C(COP(=O)([O-])OP(=O)([O-])OP(=O)([O-])[O-])O2)cc1I] |
| 5-Fluoro-dUTP | 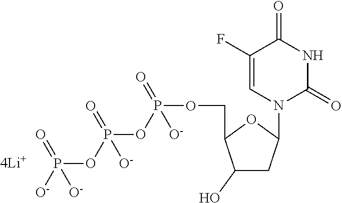 [SMILES: O=c1[nH]c(=O)n(C2CC(O)C(COP(=O)([O-])OP(=O)([O-])OP(=O)([O-])[O-])O2)cc1F] |
| dUTP | 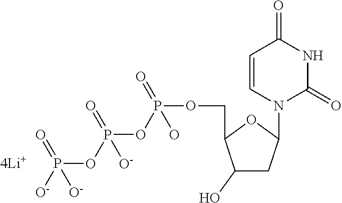 [SMILES: O=c1ccn(C2CC(O)C(COP(=O)(O)OP(=O)([O-])OP(=O)([O-])[O-])O2)c(=O)[nH]1] |
| 5-Propynyl- dUTP | 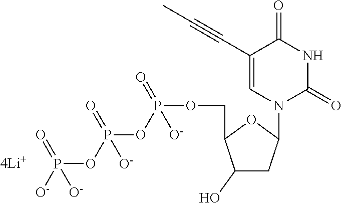 [SMILES: CC#Cc1cn(C2CC(O)C(COP(=O)([O-])OP(=O)([O-])OP(=O)([O-])[O-])O2)c(=O)[nH]c1=O] |
| 5-Propynyl- dCTP | 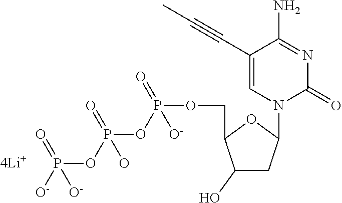 [SMILES: CC#Cc1cn(C2CC(O)C(COP(=O)([O-])OP(=O)(O)OP(=O)([O-])[O-])O2)c(=O)nc1N] |
| dITP | 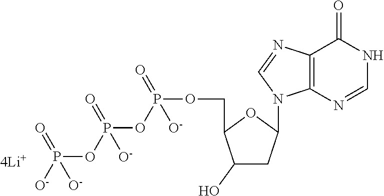 [SMILES: O=c1[nH]cnc2c1ncn2C1CC(O)C(COP(=O)([O-])OP(=O)([O-])OP(=O)([O-])[O-])O1] |
| 7-Deaza-dGTP | 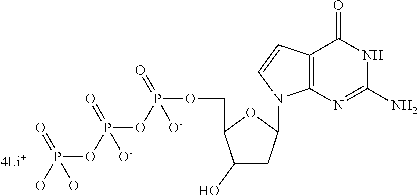 [SMILES: Nc1nc2c(ccn2C2CC(O)C(COP(=O)([O-])OP(=O)([O-])OP(=O)(O)O)O2)c(=O)[nH]1] |
| 6-Cl-purine- drTP | 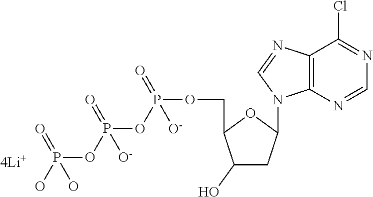 [SMILES: O=P(O)(O)OP(=O)([O-])OP(=O)([O-])OCC1OC(n2cnc3c(Cl)ncnc32)CC1O] |
| 5-Br-dUTP | 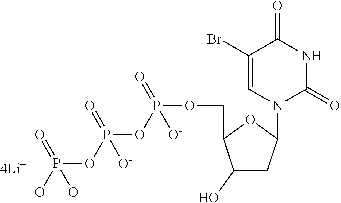 [SMILES: O=c1[nH]c(=O)n(C2CC(O)C(COP(=O)([O-])OP(=O)([O-])OP(=O)(O)O)O2)cc1Br] |
| 5-Br-dCTP | 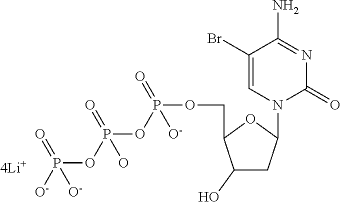 [SMILES: Nc1nc(=O)n(C2CC(O)C(COP(=O)([O-])OP(=O)(O)OP(=O)([O-])[O-])O2)cc1Br] |
| 2-Amino- purine-drTP | 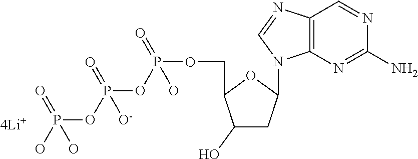 [SMILES: Nc1ncc2ncn(C3CC(O)C(COP(=O)(O)OP(=O)([O-])OP(=O)(O)O)O3)c2n1] |
| 2-Amino- dATP | 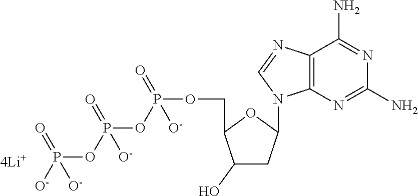 [SMILES: Nc1nc(N)c2ncn(C3CC(O)C(COP(=O)([O-])OP(=O)([O-])OP(=O)([O-])[O-])O3)c2n1] |
| 2-Amino-6-Cl- purine-drTP | 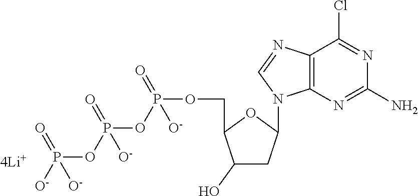 [SMILES: Nc1nc(Cl)c2ncn(C3CC(O)C(COP(=O)([O-])OP(=O)([O-])OP(=O)([O-])[O-])O3)c2n1] |
| N¹-Propyl- Pseudo-UTP | 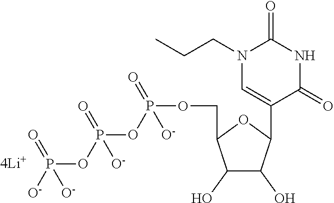 [SMILES: CCCn1cc(C2OC(COP(=O)([O-])OP(=O)([O-])OP(=O)([O-])[O-])C(O)C2O)c(=O)[nH]c1=O] |
| N¹-MOM- Pseudo-UTP | 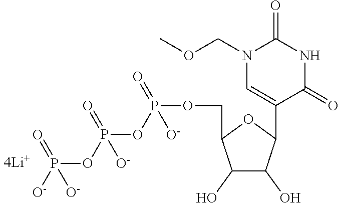 [SMILES: COCn1cc(C2OC(COP(=O)([O-])OP(=O)([O-])OP(=O)([O-])[O-])C(O)C2O)c(=O)[nH]c1=O] |
| N¹-Ethyl- pseudo-UTP | 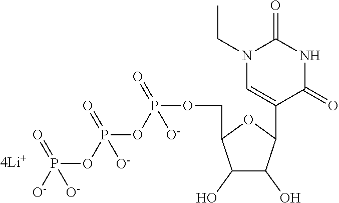 [SMILES: CCn1cc(C2OC(COP(=O)([O-])OP(=O)([O-])OP(=O)([O-])[O-])C(O)C2O)c(=O)[nH]c1=O] |
| Ara-GTP | 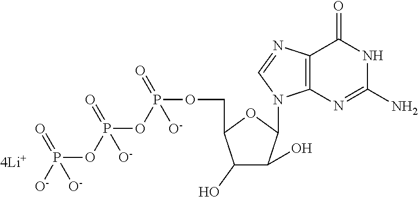 [SMILES: Nc1nc2c(ncn2C2OC(COP(=O)([O-])OP(=O)([O-])OP(=O)([O-])[O-])C(O)C2O)c(=O)[nH]1] |
| Iso-GTP | 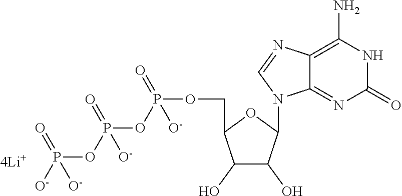 [SMILES: Nc1[nH]c(=O)nc2c1ncn2C1OC(COP(=O)([O-])OP(=O)([O-])OP(=O)([O-])[O-])C(O)C1O] |
| 8-Oxo-ATP | 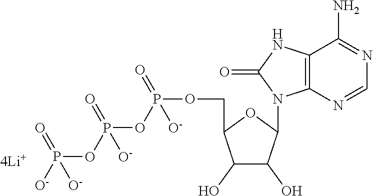 [SMILES: Nc1ncnc2c1[nH]c(=O)n2C1OC(COP(=O)([O-])OP(=O)([O-])OP(=O)([O-])[O-])C(O)C1O] |
| Thieno-CTP | 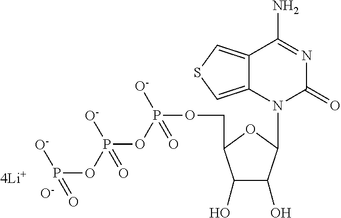 |
| 5-Carboxy- methylester- UTP | 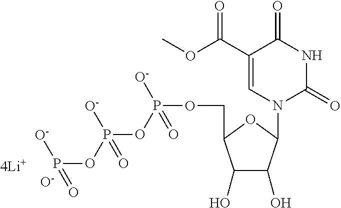 |
| Thieno-UTP | 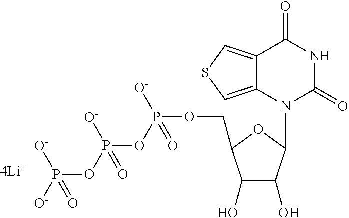 [SMILES: O=c1[nH]c(=O)n(C2OC(COP(=O)([O-])OP(=O)([O-])OP(=O)([O-])[O-])C(O)C2O)c2cscc12] |
| 5-Methoxy- CTP | 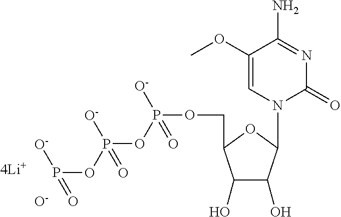 |
| 5-Methoxy- UTP | 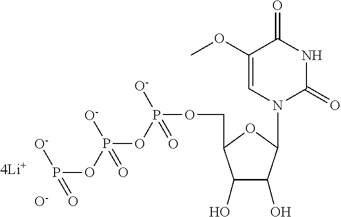 |
| 5-Hydroxy- UTP | 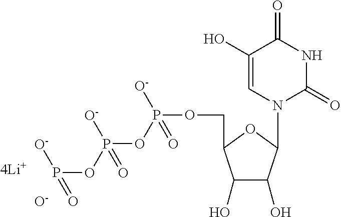 |
| 5-Carboxy- UTP | 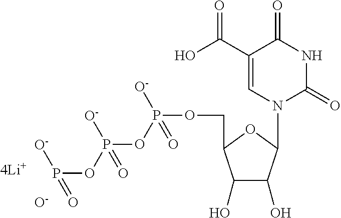 |
| 5-Formyl- UTP | 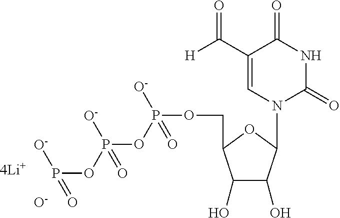 |
| 5-Hydroxy- CTP | 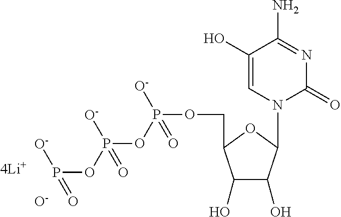 |
| Thieno-GTP | 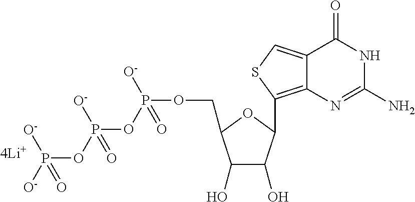 [SMILES: Nc1nc2c(C3OC(COP(=O)([O-])OP(=O)([O-])OP(=O)([O-])[O-])C(O)C3O)scc2c(=O)[nH]1] |
| 5-Hydroxy- methyl-CTP | 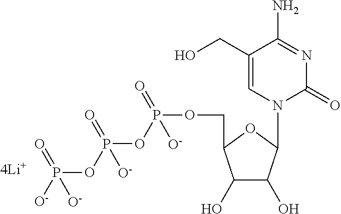 [SMILES: Nc1nc(=O)n(C2OC(COP(=O)([O-])OP(=O)([O-])OP(=O)([O-])[O-])C(O)C2O)cc1CO] |
| 5-Hydroxy- methyl-UTP | 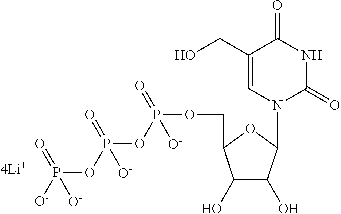 [SMILES: O=c1[nH]c(=O)n(C2OC(COP(=O)([O-])OP(=O)([O-])OP(=O)([O-])[O-])C(O)C2O)cc1CO] |
| 5-Formyl-CTP | 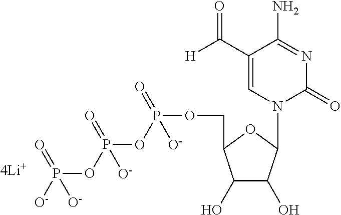 [SMILES: Nc1nc(=O)n(C2OC(COP(=O)([O-])OP(=O)([O-])OP(=O)([O-])[O-])C(O)C2O)cc1C=O] |
| N6-Methyl- Amino-ATP | 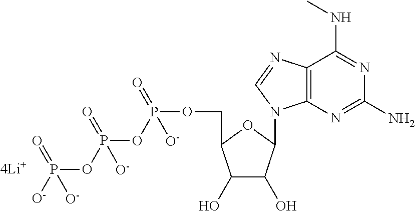 [SMILES: CNc1nc(N)nc2c1ncn2C1OC(COP(=O)([O-])OP(=O)([O-])OP(=O)([O-])[O-])C(O)C1O] |
| 5,6-Dihydro-5- Me-UTP | 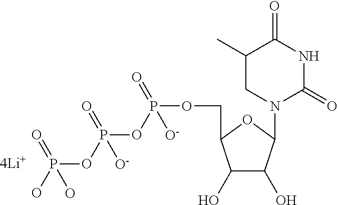 [SMILES: CC1CN(C2OC(COP(=O)([O-])OP(=O)([O-])OP(=O)(O)O)C(O)C2O)C(=O)NC1=O] |
| m¹ΨTP | 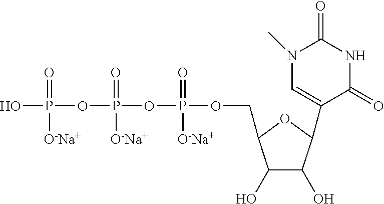 |
| N⁴-Methyl- CTP | 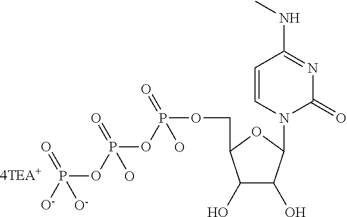 [SMILES: CNc1ccn(C2OC(COP(=O)(O)OP(=O)(O)OP(=O)([O-])[O-])C(O)C2O)c(=O)n1] |
| 2-Amino- purine-rTP | 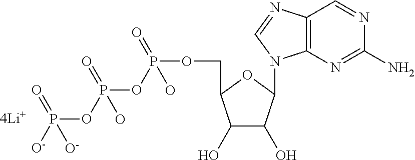 [SMILES: Nc1ncc2ncn(C3OC(COP(=O)(O)OP(=O)(O)OP(=O)([O-])[O-])C(O)C3O)c2n1] |
| 8-Oxo-GTP | 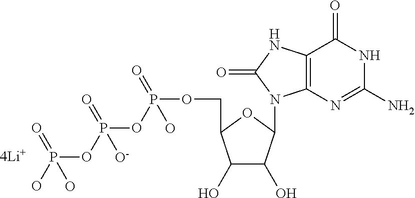 [SMILES: Nc1nc2c([nH]c(=O)n2C2OC(COP(=O)(O)OP(=O)([O-])OP(=O)(O)O)C(O)C2O)c(=O)[nH]1] |
| 5-AA-CTP | 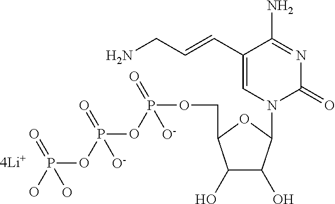 [SMILES: NC/C=C/c1cn(C2OC(COP(=O)([O-])OP(=O)([O-])OP(=O)(O)O)C(O)C2O)c(=O)nc1N] |
| 5-AA-UTP | 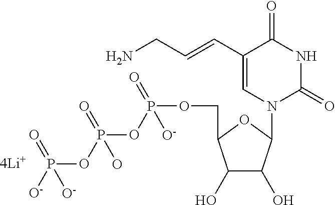 [SMILES: NC/C=C/c1cn(C2OC(COP(=O)([O-])OP(=O)(O)OP(=O)([O-])[O-])C(O)C2O)c(=O)[nH]c1=O] |
| 5-Br-UTP | 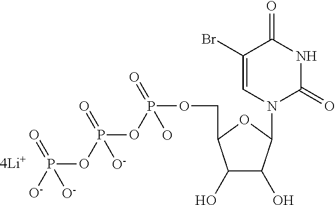 [SMILES: O=c1[nH]c(=O)n(C2OC(COP(=O)(O)OP(=O)([O-])OP(=O)([O-])[O-])C(O)C2O)cc1Br] |
| 5-Br-CTP | 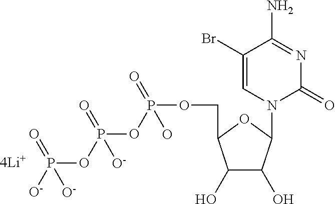 [SMILES: Nc1nc(=O)n(C2OC(COP(=O)(O)OP(=O)([O-])OP(=O)([O-])[O-])C(O)C2O)cc1Br] |
| 8-Azido-ATP | 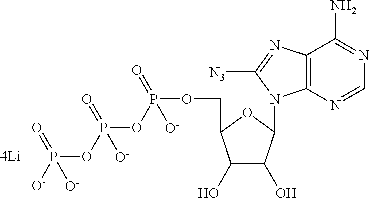 [SMILES: [N-]=[N+]=Nc1nc2c(N)ncnc2n1C1OC(COP(=O)([O-])OP(=O)([O-])OP(=O)([O-])[O-])C(O)C1O] |
| 7-Deaza-GTP | 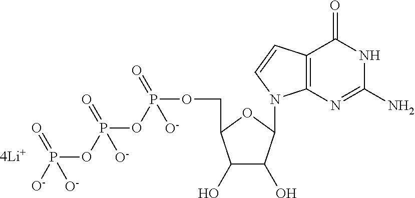 [SMILES: Nc1nc2c(ccn2C2OC(COP(=O)([O-])OP(=O)([O-])OP(=O)([O-])[O-])C(O)C2O)c(=O)[nH]1] |
| N¹-Methyl- ATP | 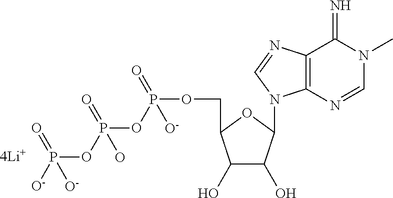 [SMILES: Cn1cnc2c(ncn2C2OC(COP(=O)([O-])OP(=O)(O)OP(=O)([O-])[O-])C(O)C2O)c1=N] |
| 6-Aza-UTP | 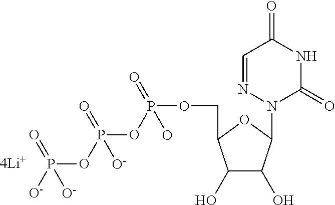 [SMILES: O=c1cnn(C2OC(COP(=O)(O)OP(=O)([O-])OP(=O)([O-])[O-])C(O)C2O)c(=O)[nH]1] |
| 6-Aza-CTP | 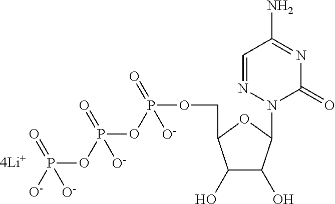 [SMILES: Nc1cnn(C2OC(COP(=O)([O-])OP(=O)([O-])OP(=O)([O-])[O-])C(O)C2O)c(=O)n1] |
| 2-Thio-CTP | 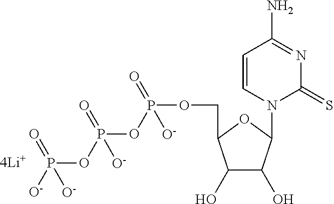 [SMILES: Nc1ccn(C2OC(COP(=O)([O-])OP(=O)([O-])OP(=O)([O-])[O-])C(O)C2O)c(=S)n1] |
| 5,6-Dihydro- UTP | 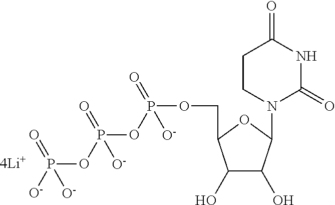 [SMILES: O=C1CCN(C2OC(COP(=O)([O-])OP(=O)([O-])OP(=O)([O-])[O-])C(O)C2O)C(=O)N1] |
| 2-Thio-UTP | 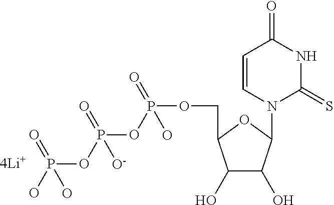 [SMILES: O=c1ccn(C2OC(COP(=O)(O)OP(=O)([O-])OP(=O)(O)O)C(O)C2O)c(=S)[nH]1] |
| O⁶-Methyl- GTP | 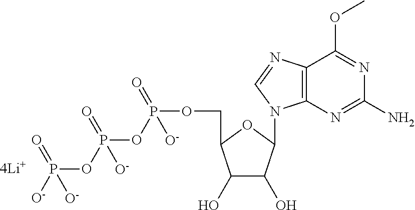 [SMILES: COc1nc(N)nc2c1ncn2C1OC(COP(=O)([O-])OP(=O)([O-])OP(=O)([O-])[O-])C(O)C1O] |
| 4-Thio-UTP | 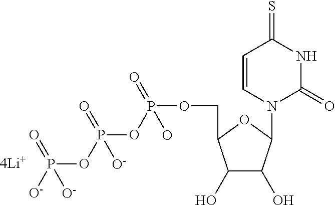 [SMILES: O=c1[nH]c(=S)ccn1C1OC(COP(=O)(O)OP(=O)([O-])OP(=O)([O-])[O-])C(O)C1O] |
| 5-Methyl-UTP | 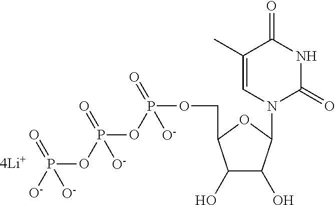 [SMILES: Cc1cn(C2OC(COP(=O)([O-])OP(=O)([O-])OP(=O)([O-])[O-])C(O)C2O)c(=O)[nH]c1=O] |
| Xanthosine- TP | 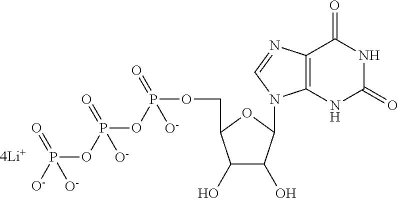 [SMILES: O=c1[nH]c(=O)c2ncn(C3OC(COP(=O)([O-])OP(=O)([O-])OP(=O)([O-])[O-])C(O)C3O)c2[nH]1] |
| ITP | 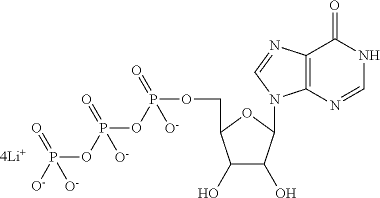 [SMILES: O=c1[nH]cnc2c1ncn2C1OC(COP(=O)([O-])OP(=O)([O-])OP(=O)([O-])[O-])C(O)C1O] |
| 5-Iodo-UTP | 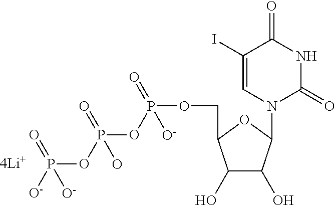 [SMILES: O=c1[nH]c(=O)n(C2OC(COP(=O)([O-])OP(=O)(O)OP(=O)([O-])[O-])C(O)C2O)cc1I] |
| 5-Iodo-CTP | 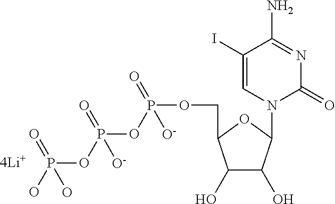 [SMILES: Nc1nc(=O)n(C2OC(COP(=O)([O-])OP(=O)([O-])OP(=O)(O)O)C(O)C2O)cc1I] |
| 6-Cl-purine- rTP | 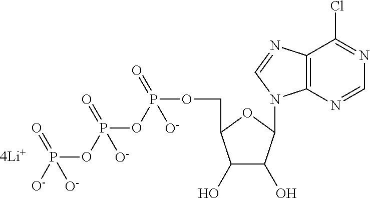 [SMILES: O=P([O-])([O-])OP(=O)([O-])OP(=O)([O-])OCC1OC(n2cnc3c(Cl)ncnc32)C(O)C1O] |

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, in the examples above, the RNA molecules (e.g. model RNAs) in the initial sample are or contain mRNA molecules. In some other examples, the RNA molecules in the initial sample can be one or more of transfer RNA (tRNA), ribosomal RNA (rRNA), short interfering RNA (siRNA), short-hairpin RNA (shRNA), micro-RNA (miRNA), an isolated or synthetic RNA oligonucleotide. The initial sample may or may not contain RNA, and any other molecules.

What is claimed is:

1. A method for preparing a sample comprising one or more RNA molecules, comprising the steps of:
i) reacting the sample with a reaction mixture comprising a poly(A) polymerase and one or more predefined nucleotide analogues to add a first sequence comprising one or more predefined nucleotide analogues to individual 3′-end of the one or more RNA molecules, such that one or more first tailed RNA is formed; and
ii) adding a plurality of adenosine triphosphates (ATP) to the reaction mixture, such that the one or more first tailed RNA is reacted with the plurality of adenosine triphosphates (ATP) to add a second sequence comprising a plurality of adenine nucleotides to individual 3′-end of the one or more first tailed RNA, such that one or more second tailed RNA is formed.

2. The method of claim 1, wherein the one or more predefined nucleotide analogues in the reaction mixture comprises one or more predefined nucleoside analogue triphosphates selected from the group consisting of 2-Amino-ATP, 2-Amino-6-Cl-purine-rTP, 2′-F-dATP, 2′-Azido-dATP, 7-Deaza-ATP, 2-Cl-ATP, 2′-Bromo-dATP, 2-Fluoro-ATP, 8-Bromo-ATP, 8-Iodo-ATP, 2′-Iodo-dATP, 1-Thio-UTP, 1-Thio-GTP, 1-Thio-CTP, 1-Thio-ATP, 1-Thio-dTTP, 1-Thio-dGTP, 1-Thio-dCTP, 1-Thio-dATP, 5-(Cyanine 7)-UTP, Cyanine 5-UTP, 6-(Cyanine 5)-CTP, 6-(Cyanine 3)-UTP, 6-(Cyanine 3)-CTP, Cyanine 5-dUTP, Cyanine 5-dCTP, Cyanine 3-dUTP, Cyanine 3-dCTP, Biotin-16-7-Deaza-dGTP, Desthiobiotin-16-UTP, Desthiobiotin-6-dCTP, Dabcyl-5-3-Aminoallyl-2′-dUTP, Biotin-16-AA-UTP, N⁴-Biotin-dCTP, Biotin-16-AA-CTP, Biotin-16-AA-dCTP, Biotin-16-AA-dUTP, dTTP, dGTP, dCTP, dATP, 7-Deaza-7-Propargylamino-dGTP, 7-Deaza-7-Propargylamino-dATP, 5-Formyl-dUTP, 5-cadUTP, 5-Indolyl-AA-dUTP, 5-Formyl-dCTP, 5-Propargylamino-dUTP, 5-Propargylamino-dCTP, 5-Hydroxymethyl-dUTP, 2′-Deoxyzebularine-TP, N⁴-Methyl-dCTP, 5-AA-dUTP, 5-AA-dCTP, 8-Chloro-dATP, 6-Thio-dGTP, 6-Aza-dUTP, 2-Thio-dCTP, 4-Thio-dTTP, 5-Hydroxy-dCTP, dPTP, 2-Thio-dTTP, 8-Oxo-Methyl-dCTP, N⁶-Methyl-dATP, 5-Iodo-dUTP, 5-Iodo-dCTP, 5-Fluoro-dUTP, dUTP, 5-Propynyl-dUTP, 5-Propynyl-dCTP, dITP, 7-Deaza-dGTP, 6-Cl-purine-drTP, 5-Br-dUTP, 5-Br-dCTP, 2-Aminopurine-drTP, 2-Amino-dATP, 2-Amino-6-Cl-purine-drTP, N¹-Propyl-Pseudo-UTP, N¹-MOM-Pseudo-UTP, N¹-Ethylpseudo-UTP, Ara-GTP, Iso-GTP, 8-Oxo-ATP, Thieno-CTP, 5-Carboxymethylester-UTP, Thieno-UTP, 5-Methoxy-CTP, 5-Methoxy-UTP, 5-Hydroxy-UTP, 5-Carboxy-UTP, 5-Formyl-UTP, 5-Hydroxy-CTP, Thieno-GTP, 5-Hydroxymethyl-CTP, 5-Hydroxymethyl-UTP, 5-Formyl-CTP, N⁶-Methyl-Amino-ATP, 5,6-Dihydro-5-Me-UTP, m¹ΨTP, N⁴-Methyl-CTP, 2-Aminopurine-rTP, 8-Oxo-GTP, 5-AA-CTP, 5-AA-UTP, 5-Br-UTP, 5-Br-CTP, 8-Azido-ATP, 7-Deaza-GTP, N¹-Methyl-ATP, 6-Aza-UTP, 6-Aza-CTP, 2-Thio-CTP, 5,6-Dihydro-UTP, 2-Thio-UTP, O⁶-Methyl-GTP, 4-Thio-UTP, 5-Methyl-UTP, Xanthosine-TP, ITP, 5-Iodo-UTP, 5-Iodo-CTP, 6-Cl-purine-rTP, 8-Aza-ATP, and combination thereof.

3. The method of claim 2, wherein the one or more predefined nucleoside analogue triphosphates are selected from the group consisting of 2-Amino-ATP, 2-Amino-6-Cl-purine-rTP, 2′-F-dATP, 2′-Azido-dATP, and 7-Deaza-ATP, and combination thereof.

4. The method of claim 1, wherein the poly(A) polymerase is a wild type E. coli poly(A) polymerase, or a mutant thereof, or a homolog thereof.

5. The method of claim 1, wherein the second sequence comprises at least 10 adenine nucleotides.

6. The method of claim 2, wherein the one or more predefined nucleoside analogue triphosphates are 2-Amino-6-Cl-purine-rTP, and step (i) further includes the following step:
incubating the sample with the reaction mixture for a predetermined time of at least 5 minutes.

7. The method of claim 6, wherein the predetermined time is about 10-20 minutes.

8. A method for sample preparation for direct RNA sequencing, comprising the steps of:
i) reacting the sample comprising one or more RNA molecules with a reaction mixture comprising a poly(A) polymerase and one or more predefined nucleotide analogues to add a first sequence comprising one or more predefined nucleotide analogues to individual 3′-end of the one or more RNA molecules, such that one or more first tailed RNA is formed; and
ii) adding a plurality of adenosine triphosphates (ATP) to the reaction mixture, such that the one or more first tailed RNA is reacted with the plurality of adenosine triphosphates (ATP) to add a second sequence comprising a plurality of adenine nucleotides to individual 3′-end of the one or more first tailed RNA, such that one or more second tailed RNA is formed.

9. The method of claim 8, wherein the method further comprises the following steps:
iii) adding a sequencing adapter and a ligase to the reaction mixture, such that the sequencing adapter is ligated to the one or more second tailed RNA, such that one or more ligated RNA is formed; and
iv) purifying the one or more ligated RNA from the reaction mixture.

10. The method of claim 9, wherein the sequencing adapter comprises an oligo-(dT) overhang.

11. The method of claim 8, wherein the one or more predefined nucleotide analogues in the reaction mixture comprises one or more predefined nucleoside analogue triphosphates selected from the group consisting of 2-Amino-ATP, 2-Amino-6-Cl-purine-rTP, 2′-F-dATP, 2′-Azido-dATP, 7-Deaza-ATP, 2-Cl-ATP, 2′-Bromo-dATP, 2-Fluoro-ATP, 8-Bromo-ATP, 8-Iodo-ATP, 2′-Iodo-dATP, 1-Thio-UTP, 1-Thio-GTP, 1-Thio-CTP, 1-Thio-ATP, 1-Thio-dTTP, 1-Thio-dGTP, 1-Thio-dCTP, 1-Thio-dATP, 5-(Cyanine 7)-UTP, Cyanine 5-UTP, 6-(Cyanine 5)-CTP, 6-(Cyanine 3)-UTP, 6-(Cyanine 3)-CTP, Cyanine 5-dUTP, Cyanine 5-dCTP, Cyanine 3-dUTP, Cyanine 3-dCTP, Biotin-16-7-Deaza-dGTP, Desthiobiotin-16-UTP, Desthiobiotin-6-dCTP, Dabcyl-5-3-Aminoallyl-2′-dUTP, Biotin-16-AA-UTP, N⁴-Biotin-dCTP, Biotin-16-AA-CTP, Biotin-16-AA-dCTP, Biotin-16-AA-dUTP, dTTP, dGTP, dCTP, dATP, 7-Deaza-7-Propargylamino-dGTP, 7-Deaza-7-Propargylamino-dATP, 5-Formyl-dUTP, 5-cadUTP, 5-Indolyl-AA-dUTP, 5-Formyl-dCTP, 5-Propargylamino-dUTP, 5-Propargylamino-dCTP, 5-Hydroxymethyl-dUTP, 2′-Deoxyzebularine-TP, N⁴-Methyl-dCTP, 5-AA-dUTP, 5-AA-dCTP, 8-Chloro-dATP, 6-Thio-dGTP, 6-Aza-dUTP, 2-Thio-dCTP, 4-Thio-dTTP, 5-Hydroxy-dCTP, dPTP, 2-Thio-dTTP, 8-Oxo-Methyl-dCTP, No-Methyl-dATP, 5-Iodo-dUTP, 5-Iodo-dCTP, 5-Fluoro-dUTP, dUTP, 5-Propynyl-dUTP, 5-Propynyl-dCTP, dITP, 7-Deaza-dGTP, 6-Cl-purine-drTP, 5-Br-dUTP, 5-Br-dCTP, 2-Aminopurine-drTP, 2-Amino-dATP, 2-Amino-6-Cl-purine-drTP, N¹-Propyl-Pseudo-UTP, N¹-MOM-Pseudo-UTP, N¹-Ethylpseudo-UTP, Ara-GTP, Iso-GTP, 8-Oxo-ATP, Thieno-CTP, 5-Carboxymethylester-UTP, Thieno-UTP, 5-Methoxy-CTP, 5-Methoxy-UTP, 5-Hydroxy-UTP, 5-Carboxy-UTP, 5-Formyl-UTP, 5-Hydroxy-CTP, Thieno-GTP, 5-Hydroxymethyl-CTP, 5-Hydroxymethyl-UTP, 5-Formyl-CTP, N⁶-Methyl-Amino-ATP, 5,6-Dihydro-5-Me-UTP, m¹ΨTP, N⁴-Methyl-CTP, 2-Aminopurine-rTP, 8-Oxo-GTP, 5-AA-CTP, 5-AA-UTP, 5-Br-UTP, 5-Br-CTP, 8-Azido-ATP, 7-Deaza-GTP, N¹-Methyl-ATP, 6-Aza-UTP, 6-Aza-CTP, 2-Thio-CTP, 5,6-Dihydro-UTP, 2-Thio-UTP, O⁶-Methyl-GTP, 4-Thio-UTP, 5-Methyl-UTP, Xanthosine-TP, ITP, 5-Iodo-UTP, 5-Iodo-CTP, 6-Cl-purine-rTP, 8-Aza-ATP, and combination thereof.

12. The method of claim 11, wherein the one or more predefined nucleoside analogue triphosphates are selected from the group consisting of 2-Amino-ATP, 2-Amino-6-Cl-purine-rTP, 2′-F-dATP, 2′-Azido-dATP, and 7-Deaza-ATP, and combination thereof.

13. The method of claim 8, wherein the poly(A) polymerase is a wild type E. coli poly(A) polymerase, or a mutant thereof, or a homolog thereof.

14. The method of claim 8, wherein the second sequence comprises at least 10 adenine nucleotides.

15. The method of claim 8, wherein the one or more predefined nucleoside analogue triphosphates are 2-Amino-6-Cl-purine-rTP, and step (i) further includes the following step:
incubating the sample with the reaction mixture for a predetermined time of at least 5 minutes.

16. The method of claim 15, wherein the predetermined time is about 10-20 minutes.

17. A kit for preparing a sample comprising one or more RNA molecules for sequencing, comprising:
a) a poly(A) polymerase;
b) one or more predefined nucleotide analogues; and
c) a plurality of adenosine triphosphates (ATP),
and optionally,
d) a ligase;
e) a sequencing adapter; and/or
f) one or more buffer solutions.

18. The kit of claim 17, further comprises:
instructions for preparing the sample comprising one or more RNA molecules by the following steps:
i) reacting the sample with a reaction mixture comprising the poly(A) polymerase and the one or more predefined nucleotide analogues to add a first sequence comprising one or more predefined nucleotide analogues to individual 3′-end of the one or more RNA molecules, such that one or more first tailed RNA is formed; and
ii) adding a plurality of adenosine triphosphates (ATP) to the reaction mixture, such that the one or more first tailed RNA is reacted with the plurality of adenosine triphosphates (ATP) to add a second sequence comprising a plurality of adenine nucleotides to individual 3′-end of the one or more first tailed RNA, such that one or more second tailed RNA is formed.

19. The kit of claim 17, wherein the one or more predefined nucleotide analogues comprises one or more nucleoside analogue triphosphates selected from the group consisting of 2-Amino-ATP, 2-Amino-6-Cl-purine-rTP, 2′-F-dATP, 2′-Azido-dATP, 7-Deaza-ATP, 2-Cl-ATP, 2′-Bromo-dATP, 2-Fluoro-ATP, 8-Bromo-ATP, 8-Iodo-ATP, 2′-Iodo-dATP, 1-Thio-UTP, 1-Thio-GTP, 1-Thio-CTP, 1-Thio-ATP, 1-Thio-dTTP, 1-Thio-dGTP, 1-Thio-dCTP, 1-Thio-dATP, 5-(Cyanine 7)-UTP, Cyanine 5-UTP, 6-(Cyanine 5)-CTP, 6-(Cyanine 3)-UTP, 6-(Cyanine 3)-CTP, Cyanine 5-dUTP, Cyanine 5-dCTP, Cyanine 3-dUTP, Cyanine 3-dCTP, Biotin-16-7-Deaza-dGTP, Desthiobiotin-16-UTP, Desthiobiotin-6-dCTP, Dabcyl-5-3-Aminoallyl-2′-dUTP, Biotin-16-AA-UTP, N⁴-Biotin-dCTP, Biotin-16-AA-CTP, Biotin-16-AA-dCTP, Biotin-16-AA-dUTP, dTTP, dGTP, dCTP, dATP, 7-Deaza-7-Propargylamino-dGTP, 7-Deaza-7-Propargylamino-dATP, 5-Formyl-dUTP, 5-cadUTP, 5-Indolyl-AA-dUTP, 5-Formyl-dCTP, 5-Propargylamino-dUTP, 5-Propargylamino-dCTP, 5-Hydroxymethyl-dUTP, 2′-Deoxyzebularine-TP, N⁴-Methyl-dCTP, 5-AA-dUTP, 5-AA-dCTP, 8-Chloro-dATP, 6-Thio-dGTP, 6-Aza-dUTP, 2-Thio-dCTP, 4-Thio-dTTP, 5-Hydroxy-dCTP, dPTP, 2-Thio-dTTP, 8-Oxo-dGTP, 8-Oxo-dATP, 5-Nitro-1-Indolyl-drTP, N²-Methyl-dGTP, O⁶-Methyl-dGTP, 5-Methyl-dCTP, N⁶-Methyl-dATP, 5-Iodo-dUTP, 5-Iodo-dCTP, 5-Fluoro-dUTP, dUTP, 5-Propynyl-dUTP, 5-Propynyl-dCTP, dITP, 7-Deaza-dGTP, 6-Cl-purine-drTP, 5-Br-dUTP, 5-Br-dCTP, 2-Aminopurine-drTP, 2-Amino-dATP, 2-Amino-6-Cl-purine-drTP, N¹-Propyl-Pseudo-UTP, N¹-MOM-Pseudo-UTP, N¹-Ethylpseudo-UTP, Ara-GTP, Iso-GTP, 8-Oxo-ATP, Thieno-CTP, 5-Carboxymethylester-UTP, Thieno-UTP, 5-Methoxy-CTP, 5-Methoxy-UTP, 5-Hydroxy-UTP, 5-Carboxy-UTP, 5-Formyl-UTP, 5-Hydroxy-CTP, Thieno-GTP, 5-Hydroxymethyl-CTP, 5-Hydroxymethyl-UTP, 5-Formyl-CTP, N⁶-Methyl-Amino-ATP, 5,6-Dihydro-5-Me-UTP, m¹ΨTP, N⁴-Methyl-CTP, 2-Aminopurine-rTP, 8-Oxo-GTP, 5-AA-CTP, 5-AA-UTP, 5-Br-UTP, 5-Br-CTP, 8-Azido-ATP, 7-Deaza-GTP, N¹-Methyl-ATP, 6-Aza-UTP, 6-Aza-CTP, 2-Thio-CTP, 5,6-Dihydro-UTP, 2-Thio-UTP, O⁶-Methyl-GTP, 4-Thio-UTP, 5-Methyl-UTP, Xanthosine-TP, ITP, 5-Iodo-UTP, 5-Iodo-CTP, 6-Cl-purine-rTP, 8-Aza-ATP, and combination thereof.

20. The kit of claim 19, wherein the one or more predefined nucleoside analogue triphosphates are selected from the group consisting of 2-Amino-ATP, 2-Amino-6-Cl-purine-rTP, 2′-F-dATP, 2′-Azido-dATP, and 7-Deaza-ATP, and combination thereof.
